(19) United States
(12) Patent Application Publication
(10) Pub. No.: US 2017/0096656 A1
(43) Pub. Date: Apr. 6, 2017

(54) THERMOSTABLE ALGINATE DEGRADING ENZYMES AND THEIR METHODS OF USE

(71) Applicants: MATIS OHF., Reykjavik (IS); RIJKSUNIVERSITEIT GRONINGEN, Groninggen (NL); UNIVERSITÄT STUTTGART, Stuttgart (DE)

(72) Inventors: Gudmundur Oli Hreggvidsson, Reykjavik (IS); Jon Oskar Jonsson Wheat, Reykjavik (IS); Bryndis Bjornsdottir, Reykjavik (IS); Olafur Hedinn Fridjonsson, Reykjavik (IS); Josef Altenbuchner, Stuttgart (DE); Hildegard Watzlawick, Stuttgart (DE); Justyna M. Dobruchowska, Groningen (NL); Johannis P. Kamerling, Groningen (NL)

(21) Appl. No.: 15/110,132

(22) PCT Filed: Jan. 12, 2015
(86) PCT No.: PCT/IS2015/050001

§ 371 Date: Oct. 3, 2016

Related U.S. Application Data
(60) Provisional application No. 61/926,009, filed on Jan. 10, 2014.

(51) Int. Cl. C12N 9/88; C12P 19/02

(52) U.S. Cl. CPC C12N 9/88; C12P 19/02

(57) ABSTRACT

The present invention relates to the identification, production and use of thermostable alginate lyase enzymes that can be used to partially degrade alginate to yield oligosaccharides or to give complete degradation of alginate to yield (unsaturated) mono-uronates.

### BACKGROUND OF THE INVENTION

Macroalgae (seaweeds) are used for various industrial applications and are currently produced on large scale in various parts of the world. The biomass production per area of macroalgae has been estimated to be up to 2.8 times higher than of sugarcane [1]. This production is likely to increase in the future, since their growth rate also increases with higher CO₂ concentration and global warming. Macroalgae are therefore also a promising non-food feedstock for bioconversion into 2^{nd} generation biofuels.

Brown algae (Phaeophyceae) are very promising biorefinery feedstock species because of the potential high bulk biomass production. The major constituent carbohydrates of Brown algae, i.e. alginate, laminarin and mannitol, amount up to 60% of dry weight in common Brown algae such as Laminaria. Alginate constitutes more than ⅓ of the carbohydrates and needs to be utilized to ensure cost effective production of biofuels and/or platform chemicals (such as organic acids and diols) from macroalgal biomass by microbial fermentation. Efficient conversion of alginate to fermentable (unsaturated) monosaccharides is therefore essential. As an example with alginate utilization included, the total ethanol yields per biomass dry weight of Brown algae will be significantly higher for seaweed than for lignocellulose [2].

Alginate (alginic acid) is a major constituent of the algal cell wall and intracellular material. It is a linear polymer comprised of blocks of (1→4)-linked β-D-mannuronate (M) and its (1→4)-linked C-5 epimer α-L-guluronate (G) residues. The residues are arranged in homopolymeric blocks of consecutive M- or G-residues, or heteropolymeric blocks of alternating M and G-residues [reviewed in 3]. Two genera of bacteria, Pseudomonas and Azotobacter, are known to produce alginate as exopolysaccharide [4, 5]. Alginate occurs as a Ca²⁺-alginate gel in the seaweed. Aqueous alginate solutions are therefore highly viscous and the polysaccharides are not easily accessible for enzymatic degradation. Replacement of Ca²⁺ with Na⁺, e.g. by adding large excess of Na⁺, will dissolve alginate and metal chelators (e.g. EDTA) that will bind Ca²⁺ have been applied for cell wall degradation in combination with enzymatic disruption/eliminative cleavage by alginate lyases [6]. However, this is not economically feasible on an industrial scale.

Alginate is an important industrial polysaccharide and has a wide variety of uses in the food sector for its dehydrating, gelling, stabilizing and thickening properties [reviewed in 7], as well as in various biotechnological and medical applications [reviewed in 3, 8]. The degradation of alginate to oligosaccharides and further to (unsaturated) mono-uronates is a challenging task on an industrial scale. Alginate is very resistant to acid hydrolysis and chemicals in the quantities needed for efficient industrial hydrolysis and their subsequent removal are costly.

Enzymatic degradation of alginate can be either selective or complete depending on the choice of enzymes. Enzymatic degradation is inherently a more economical process and environmentally more benign than chemical hydrolysis. Provided that substrate specificity demands are met by the enzymes, the technological set up would be simpler as chemical processes would need additional steps of neutralization and removal of chemicals. More robust thermostable enzymes, as described in this invention, may be used directly in crude feedstock slurries, and high process temperature would be advantageous leading to greater solubilisation of alginate, reduce viscosity and correspondingly facilitate enzymatic access to the polysaccharide chain.

For efficient degradation of alginate, enzymes of different specificities with respect to MM, GG and MG/GM blocks are needed. Alginate degrading enzymes have been identified from various organisms, including alginate utilizing bacteria. Alginate lyases catalyse β-elimination of the 4-O glycosidic bond between monomers, forming a double bond between the C4 and C5 carbons. Classification is based on their substrate specificity towards cleavage of M-rich blocks (polyM lyases), G-rich blocks (polyG lyases), or heteropolymeric MG blocks (polyMG lyases). The enzymes therefore typically have high specific activity towards one type of bond, with much lower, or no activity toward the other bond types, whereby also the specific microenvironment plays a role. Therefore no single alginate lyase enzyme is known to show high enough cleavage activity on all bond types, in order to give substantially complete degradation of alginate, with high conversion into (unsaturated) mono-uronates. In the present invention, we describe how near complete degradation of alginate with the production of (unsaturated) monosaccharides (mono-uronates) can be achieved with the use of a composition containing two, three or four different alginate lyase enzymes.

Several bacterial alginate lyase genes have been identified, cloned and sequenced. Both native and recombinantly expressed enzymes have been characterized, giving insight into alginate lyase structure and function [9-13]. In the Carbohydrate-Active Enzymes (CAZY) database (http://www.cazy.org/), alginate lyases are assigned to different polysaccharide lyase (PL) families based on amino acid sequence similarities. PL enzyme families may have other substrates specificities than alginate lyase activity and sequence identities between known alginate lyases can be low within a particular family. Alginate lyase activity is therefore non-obvious from primary sequence comparisons alone. As an example, AlyRm3 has seven homologues with amino acid sequence identity 22-39% with 60-90% sequence coverage (designated for clarification as the AlyRm3-group in this description). There is also a partial identity with the C-terminal region of sequences in families PL15 and PL17 (sequence coverage of around 30%) representing a Heparinase II/III-like protein domain (pfam07940 is a member of the superfamily cl15421). The apparent identity of AlyRm3 is to conserved sequence motives of the heparinase domain (see alignment in FIG. 3).

According to the comprehensive CAZY database, genes encoding polysaccharide lyases are in general rare in thermophilic bacteria, missing in most species and representative genes encoding enzymes annotated as having activity on polysaccharides containing galacturonic acid (including pectate lyases and rhamnogalacturonan lyases). Furthermore, functional annotation is often based on only few characterized enzymes in each family.

Polysaccharide lyases are classified into families (PL families) based on the similarities of their primary structure, and activity on alginate is ascribed to sequences in seven PL families, PL5, PL6, PL7, PL14, PL15, PL17, and PL18 (of 22). Representatives of these families have only been detected in genomes of four thermophilic species, in Rhodothermus marinus (PL6, PL17), Spirochaeta thermophila (PL7, PL17), Merioribacter roseus (PL6, PL17) and one particular Paenibacillus strain Y412MC10 (PL7, PL15, PL17). Four non-classified polysaccharide lyases (with unknown activity) have also been annotated as such in genomes of three thermophiles, in Caldicellulosiruptor saccharolyticus (one gene), in Meiothermus ruber (two genes) and in Thermobispora bispora (one gene). Of the four unclassified polysaccharide lyases in the CAZY database, the putative polysaccharide lyase from C. saccharolyticus has the heparinase II/III-like protein domain observed in AlyRm3 and PL15 and PL17 sequences.

A thermostable alginate lyase has been isolated and characterized from Bacillus stearothermophilus. It has optimum activity at 50° C. and is stable for 4 h at 60° C. [14]. However, it is not known to which PL family it belongs and none of the sequenced Geobacillus genomes to date (Geobacillus is the revised genus name for Bacillus stearothermophilus) harbour any identified polysaccharide lyase genes. No other thermostable alginate lyases with optimum activity higher than 60° C. have been reported so far and no such enzymes with optimum activity at 70° C. or 80° C. or higher, have been described until now. The present invention is therefore the first known discovery of such highly thermostable alginate lyases.

The four alginate lyases, AlyRm1, AlyRm2, AlyRm3 and AlyRm4, described in this invention are from Rhodothermus marinus str. 378. Two of these, AlyRm1 and AlyRm2, belong to family PL6. The three characterized enzymes to date in this family have activity on chondroitin sulfate or alginate. Two other sequences originating from a thermophile, M. roseus, are found in this family. The AlyRm1 and AlyRm2 sequences are strain specific as homologues are found in the genome of R. marinus DSM 4252 but not in the R. marinus strain SG0.5JP17-172. AlyRm4 belongs to family PL17 which contains alginate and oligoalginate lyases. It is also strain specific as homologues are only found in the R. marinus DSM 4252 genome. Other thermophilic strains containing a PL17 sequence putatively encoding alginate lyases are S. thermophila, M. roseus and the Paenibacillus strain Y412MC10. The AlyRm3 alginate lyase apparently belongs to a hitherto unknown PL family. No homologues have been detected in other thermophilic species. It is only found in R. marinus strain 378 and R. marinus DSM 4252, but not in R. marinus strain SG0.5JP17-172. Alginate lyases have valuable properties applicable for biotechnological utilisation. They have been used to determine the fine structure of alginate, for production of defined alginate oligomers, and for protoplasting seaweed [reviewed in 7]. Their application for degradation of alginate polymers produced by Pseudomonas aeruginosa in cystic fibrosis patients has been described [14] and studied [15, 16]. Alginate lyases may also be used for degradation of alginate in the production of biofuels or renewable commodity compounds from algal biomass [17, 18]. For utilization in those various industrial processes, robust alginate lyases which can function at extreme circumstances, such as elevated temperatures, may be of great value and have many uses.

The present invention relates to a new set of enzymes that extends the current scientific knowledge of alginate lyases. The present application describes the first thermophilic alginate lyases with optimum activity higher than 60° C. The genes encoding the enzymes were identified in Rhodothermus marinus str. 378, a Gram-negative, aerobic, thermophile. The bacterium, which has been isolated from marine habitats around the world in proximity to hot spring vents, is a known producer of various robust enzymes [19]. The thermophilic R. marinus alginate lyases expand the previously described activity range of alginate lyases. Higher processing temperatures are possible, which is advantageous as solubility of alginate increases and viscosity is reduced. Consequently this facilitates enzymatic access resulting in a more efficient degradation of alginate. Thermophilic enzymes also simplify integration of degradation processes with prior pre-treatment of seaweeds which is optimally carried out at elevated temperatures. Thermophilic and thermostable enzymes reduce the need for cooling from often high pre-processing temperatures of algal biomass that would be required for mesophilic enzymatic alginate degrading processes, and the associated higher costs are therefore avoided. High temperature also prevents contamination by spoilage bacteria. A further advantage of thermophilic alginate lyases is that such enzymes expressed heterologously in a mesophilic host can be purified substantially by simple and relatively inexpensive heat precipitation of the host's proteins.

The thermophilic R. marinus alginate lyases differ in activity, but together they cover a wide activity range. This allows preparation of enzyme compositions containing one, or two, or three or four such enzymes for highly selective as well for more indiscriminate degradation of alginate with regard to glycosidic bond type. Such enzyme mixtures can be used to partially degrade alginate to yield oligosaccharides composed of alginate segments consisting essentially of D-mannuronic acid residues, or segments consisting essentially of L-guluronic acid residues, and also segments consisting essentially of alternating D-mannuronic acid and L-guluronic acid residues. By controlling the composition of the enzymes also a near complete degradation of alginate to yield fermentable monomers is possible. The (unsaturated) mono-uronates can alternatively be used as substrate in chemical syntheses. Such controlled degradation to different degrees of polymerization will give products that have valuable industrial application properties.

The present invention relates to the identification, production, and use of thermostable alginate lyase enzymes, the proteins themselves and polynucleotides encoding these, which enzymes together comprise the near complete range of specificities with regard to the glycosidic bond types in GG, MM and MG/GM disaccharide units for controlled and directed degradation of alginate. They can be used according to the invention in mixtures of different compositions and proportions with regard to enzyme types and specificities. These mixtures can be optimized for either partial degradation of alginate to yield oligosaccharides of specific monomer composition or alternatively for near complete degradation to (unsaturated) monomers. The mixtures can also be optimized in enzyme composition and proportions with regard to the fractional content of different uronate blocks in respective alginate substrates which may be species dependent.

As such, the invention provides in one embodiment a recombinant construct comprising a DNA sequence comprising a coding region for alginate lyase enzyme from thermophilic bacteria, or coding for an alginate lyase active domain, as further defined herein. The presence of alginate lyases is strain specific and suitable organisms for the isolation of thermostable alginate lyase enzymes include strains belonging to the genera Alicyclobacterium, Ammonifex, Anerocellum, Anaerolinea, Anaerophaga, Anoxybacillus, Caldicellulosiruptor, Clostridium, Caldilinea, Caldisericum, Calditerrivibrio, Caloramator, Chloroacidobacterium, Carboxydothermus, Chloroflexus, Clostridium, Desulfotomaculum, Dictyoglomus, Exiguobacterium, Fervidobacterium, Geobacillus, Marinithermus, Marinitoga, Meiothermus, Oceanithermus, Paenibacillus, Petrotoga, Rhodothermus, Roseiflexus, Spirochaeta, Syntrophothermus, Thermacetogenium, Thermaerobacter, Thermanaerovibrio, Therminicola, Thermoanaerobacter, Thermoanaerobacterium, Thermobacillus, Thermobaculum, Thermobifida, Thermobispora, Thermodesulfatator, Thermodesulfobacterium, Thermodesulfobium, Thermodesulfovibrio, Thermomicrobium, Thermomonospora, Thermosediminibacter, Thermosipho, Thermosynecchococcus, Thermotoga, Thermovibrio, Thermovirga, Thermus and other thermophilic organisms.

The thermostable alginate lyase enzymes can be from R. marinus, Melioribacter roseus, Spirochaeta thermophila, Caldicellulosiruptor saccharolyticus, Meiothermus ruber, Thermobiospora biospora, Paenibacillus spp., among other organisms.

The thermostable alginate lyase enzymes can be coded by genes identical or similar to alyRm1, alyRm2, alyRm3 and alyRm4, which are disclosed herein, among other genes. The sequences of the alyRm1, alyRm2, alyRm3 and alyRm4 and useful active variants thereof genes are shown herein as SEQ ID NO:7-12, respectively.

In certain embodiments the thermostable alginate lyase enzymes can be a fusion protein that includes His-tag (His⁶) or other suitable additions.

In certain embodiments the thermostable alginate lyase enzyme is modified, such that the amino acid sequence can have a number of amino acids deletions, amino acid modifications or amino acid additions.

In certain embodiments the thermostable alginate lyase enzymes can be expressed from bacteria such as E. coli. Methods to isolate and purify the enzyme from such production system are well known to the skilled person.

In certain embodiments the thermostable alginate lyase enzymes can be expressed from eukaryotic organisms such as yeasts or fungi. The invention also encompasses certain recombinant constructs and vectors for carrying the coding sequences of the thermostable alginate lyase enzymes.

In certain embodiments a composition for use in a method of the invention comprises at least one thermostable alginate lyase enzyme, although typically a composition will comprise more enzymes, for example, two, three, four or more.

Thus, in one aspect a composition for use in the invention may comprise thermostable alginate lyase enzymes with different specificities. A composition for use in the invention may comprise more than one enzyme activity in one or more of the classes active on M-rich blocks (polyM lyases), G-rich blocks (polyG lyases), or heteropolymeric MG blocks (polyMG lyases).

In another aspect of the invention, such a composition may comprise an auxiliary enzyme activity, such as β-glucanase enzymes.

The invention also contemplates certain methods for carrying out degradation of alginate from different macroalgae, such as from Microcystis, Ascophyllum, Laminaria, Ecklonia and Sargassum, by adding an enzyme composition of the invention to solutions of the alginate and incubating the mixture at suitable temperature and pH for a suitable length of time.

In one embodiment, the alginate can be from bacteria, such as Pseudomonas and Azotobacter.

In certain methods the alginate substrates in the reaction mixture can be oligosaccharides or polysaccharides and the like or their mixtures.

In one aspect, the enzyme or enzymes in a composition for use in the invention may be derived from Rhodothermus marinus. In the invention, it is anticipated that a core set of alginate degrading enzyme activities may be derived from R. marinus. That activity can then be supplemented with additional enzyme activities from other sources. Such additional activities may be derived from classical sources and/or produced by a genetically modified organism.

In one aspect, the enzyme in a composition for use in the invention is thermostable. Herein, this means that the enzyme has a temperature optimum of 60° C. or higher, for example about 70° C. or higher, such as about 75° C. or higher, for example about 80° C. or higher, such as about 85° C. or higher. Activities in a composition for use in the invention will typically not have the same temperature optima, but preferably will, nevertheless, be thermostable.

In addition, enzyme activities in a composition for use in the invention may be able to work at neutral pH between 5 and 9. For the purposes of this invention, neutral pH indicates a pH of about 4.5 to 9.5, about 5 to 9, about 5.5 to 8.5, about 5.5 to 8, about 5.5 to 7.5, about 5.5 to 7, about 5 to 6.5, about 5 to 6, about 5.5 to 6.

Enzyme activities in a composition for use in the invention may be defined by a combination of any of the above temperature optima and pH values.

In one embodiment of the invention, different enzymes or combinations of enzymes can be used to obtain different degree of hydrolysis of alginate to unsaturated monomers.

In one embodiment of the invention, enzymes or combinations of enzymes may be immobilised, such as on a surface or in a matrix of some sort, such as in a column, in order to increase durability and reuse of the enzyme(s) during the process of alginate degradation. Immobilisation of enzymes may be one aspect of process development and may include additional aspects of enzyme manipulation for improved process design.

In one aspect, this means that by using a single alginate lyase of the invention such as AlyRm3 can give about 50% conversion into unsaturated monomers or by using an enzyme such as AlyRm4 can give about 70% conversion into unsaturated monomers.

In another aspect, this means by using a mixture of two or more enzymes of the invention, about 85% conversion into unsaturated monomers can be obtained, or such as by using a mixture of AlyRM3 and AlyRm4 together, about 99% conversion into unsaturated monomers can be obtained.

A further aspect of the invention provides an isolated polynucleotide comprising a sequence coding for a thermostable alginate lyase selected from the group consisting of AlyRm1 depicted in SEQ ID NO:1, or SEQ ID NO: 2, AlyRm2 depicted in SEQ ID NO:3, or SEQ ID NO:4, AlyRm3 depicted in SEQ ID NO:5, and AlyRm4 depicted in SEQ ID NO: 6. In certain embodiments the isolated polynucleotide comprises a sequence selected from SEQ ID NO:7,

SEQ ID NO:8, SEQ ID NO:9, SEQ ID NO:10, SEQ ID NO:11, and SEQ ID NO: 12, or a nucleotide sequence coding for the same amino acid sequence as any of these. In another embodiment, the invention provides complimentary DNA (cDNA) that codes for any of the protein(s) of the invention, such as amino acid sequences comprising the above mentioned sequences.

Additional features and advantages of the present invention are described in, and will be apparent from, the following Detailed Description of the Invention and the figures.

### BRIEF DESCRIPTION OF THE FIGURES

FIG. 1 shows the domains of the AlyRm1 and AlyRm2 alginate lyases of R. marinus. SP indicates signal peptide.

FIG. 2 shows amino acid alignments of AlyRm1 and AlyRm2 to related sequences of highest identity and all characterized enzymes in polysaccharide lyase families PL6. The N-terminal end of AlyRm2 was not included in the alignments. Insertions in sequences 10 and 11 (around position 490) were deleted for simplification (and marked as del in the figure).

FIG. 3 shows amino acid alignments of AlyRm3 and AlyRm4 to related sequences of highest identity and all characterized enzymes in polysaccharide lyase families PL15 and PL17.

FIG. 4 shows 10% SDS-PAGE of crude extracts (15 μg protein) of E. coli JM109 harbouring the respective plasmids for the alginate lyase genes and purified (His)₆-alginate lyases (3 μg protein) after IMAC. CE− non-induced crude extract; CE+ crude cell extract from rhamnose-induced cells.

FIG. 5 shows the main activity characteristics of the thermostable alginate lyase enzyme AlyRm1 from R. marinus. The variants are containing the C-terminal domain (AlyRm1) or lacking the C-terminal domain (AlyRm1ΔC). A) optimum temperature, B) optimum pH, C) thermal stability of AlyRm1, D) thermal stability of AlyRm1ΔC, E) optimum salinity.

FIG. 6 shows the main activity characteristics of the thermostable alginate lyase enzyme AlyRm2 from R. marinus. The variants are lacking the N-terminal domain (AlyRm2ΔN) or lacking both the N-terminal and C-terminal domains (AlyRm2ΔNC). A) optimum temperature, B) optimum pH, C) thermal stability of AlyRm2ΔN, D) thermal stability of AlyRm2ΔNC, E) optimum salinity.

FIG. 7 shows the main activity characteristics of the thermostable alginate lyase enzyme AlyRm3 from R. marinus.

FIG. 8 shows the main activity characteristics of the thermostable alginate lyase enzyme AlyRm4 from R. marinus.

FIG. 9 shows alginate degradation by the recombinant thermostable alginate lyase enzymes using thin layer chromatography (TLC).

FIG. 10 shows the degradation pattern after 8 h incubation of alginate (Sigma) with the thermostable alginate lyase, AlyRm1, as analyzed with 1D ¹H NMR, HPAEC-PAD and TLC.

FIG. 11 shows the degradation pattern after 8 h incubation of a G-block with the thermostable alginate lyase, AlyRm1, as analyzed with 1D ¹H NMR, HPAEC-PAD and TLC.

FIG. 12 shows the degradation pattern after 8 h incubation of an M-block with the thermostable alginate lyase, AlyRm1, as analyzed with 1D ¹H NMR, HPAEC-PAD and TLC.

FIG. 13 shows the degradation pattern after 8 h incubation of alginate (Sigma) with the thermostable alginate lyase, AlyRm2, as analyzed with 1D ¹H NMR, HPAEC-PAD and TLC.

FIG. 14 shows the degradation pattern after 8 h incubation of a G-block with the thermostable alginate lyase, AlyRm2, as analyzed with 1D ¹H NMR, HPAEC-PAD and TLC.

FIG. 15 shows the degradation pattern after 8 h incubation of an M-block with the thermostable alginate lyase, AlyRm2, as analyzed with 1D ¹H NMR, HPAEC-PAD and TLC.

FIG. 16 shows the degradation pattern after 8 h incubation of alginate (Sigma) with the thermostable alginate lyase, AlyRm3, as analyzed with 1D ¹H NMR, HPAEC-PAD and TLC.

FIG. 17 shows the degradation pattern after 8 h incubation of a G-block with the thermostable alginate lyase, AlyRm3, as analyzed with 1D ¹H NMR, HPAEC-PAD and TLC.

FIG. 18 shows the degradation pattern after 8 h incubation of an M-block with the thermostable alginate lyase, AlyRm3, as analyzed with 1D ¹H NMR, HPAEC-PAD and TLC.

FIG. 19 shows the degradation pattern after 8 h incubation of alginate (Sigma) with the thermostable alginate lyase, AlyRm4, as analyzed with 1D ¹H NMR, HPAEC-PAD and TLC.

FIG. 20 shows the degradation pattern after 8 h incubation of a G-block with the thermostable alginate lyase, AlyRm4, as analyzed with 1D ¹H NMR, HPAEC-PAD and TLC.

FIG. 21 shows the degradation pattern after 8 h incubation of an M-block with the thermostable alginate lyase, AlyRm4, as analyzed with 1D ¹H NMR, HPAEC-PAD and TLC.

### DETAILED DESCRIPTION OF THE INVENTION

Bioinformatic analysis of DNA sequence databases was used to identify the predicted open reading frame (ORF) of four alginate lyases in the genome of R. marinus isolate MAT378, termed alyRm1 to alyRm4. The enzyme domains were isolated, cloned, and expressed in soluble form in E. coli to investigate their activity. The enzymes were classified according to their domain structure. The current invention relates to thermostable/thermophilic alginate lyase enzymes from thermophilic bacteria. The recombinant enzymes were produced with polyhistidine tags that aid their purification. The enzymes were purified and their activity in degrading alginate and oligosaccharides made from β-D-mannuronic acid (M-block), or from α-L-guluronic acid (G-block) was assayed. Various assay methods were used for studying these activities and characterizing the enzymes.

In useful embodiments of the invention the lyase enzyme(s) of the invention and which are used in the methods of the invention comprise an alginate lyase domain, but parts of other sections of the full length protein as expressed native may be truncated. As described in more detail in the Examples, certain domains in the specific illustrated embodiments herein are contemplated to be lyase activity domains. Thus, the lyase domain of AlyRm1 protein can be seen as the section 20-490 aa of the full length native protein. N-terminally of this sequence is a signal peptide. In some embodiments of the invention an N-terminal signal peptide is not part of the lyase enzyme. C-terminally of the lyase domain of AlyRm1 is a section termed herein as a C-terminal attachment domain. (C-terminal part of SEQ ID:2 but not part of SEQ ID NO:1). As seen in Example X, this domain has certain effects on the activity and functional characteristics of the protein, but both variants, with and without the C-terminal domain, are active and thermophilic (with optimal activity at or above 60° C.). Accordingly, in embodiments of the invention, a lyase protein may used without such C-terminal domain, in full or in part. Thus, all methods, proteins, nucleotides and constructs disclosed and claimed herein, may in some embodiments refer to proteins and corresponding coding sequences comprising such lyase activity domain but without in full or in part such C-terminal domain and/or sequences which are natively N-terminally of the lyase activity domains such as but not limited to signal peptide sequences.

In the AlyRm2 protein, a corresponding C-terminal portion can be defined, and a protein of the invention and used in the methods of the invention may be without such C-terminal section, in full or in part. The same applies to other lyases of the invention.

In some embodiments of the invention the alginate lyase of the invention comprises a sequence selected from any of the SEQ ID NO:1, SEQ IDNO:2, SEQ ID NO:3, SEQ ID NO:4, SEQ ID NO:5, and SEQ ID NO:6. In other embodiments the alginate lyase comprises a substantially similar sequence to any of those, which retains the thermophilic alginate lyase activity. Thus, N- and/or C-terminal domains and portions may be truncated, and/or non-critical amino acids may be altered, to improve characteristics of the protein.

In general, when the description refers to herein without further specifying an ‘alginate lyase’, this term includes an alginate lyase active domain, with or without N- and/or C-terminal protein domains and sequences, that are not crucial for the lyase activity.

As illustrated in the Examples and Figures, the proteins in the exemplified embodiments of the invention have varying optimal lyase activity, ranging from about 65° C. to about 90° C. Thus in embodiments of the invention, the alginate lyase used has optimum activity at about 60° C. or higher, such as at about 65° C. or higher, and more preferably at about 70° C. or higher, or about 75° C. or higher, and more preferably at about 80° C. or higher, such as at about 85° C. or higher.

In preferred embodiments of the proteins and methods of the invention, the alginate lyase of the invention has thermostable alginate lyase activity on alginate oligo/polysaccharides such that it preferentially cleaves M-G bonds and less preferentially G-G or M-M or G-M bonds. However, in certain other embodiments, the lyase thermostable alginate lyase activity on alginate oligo/polysaccharides such that it preferentially cleaves M-G or G-G bonds and less preferentially M-M or G-M bonds. In yet further embodiments, the lyase has thermostable alginate lyase activity on alginate oligo/polysaccharides such that it preferentially cleaves M-G or G-G or M-M or G-M bonds in a random fashion. In another embodiment, the lyase thermostable alginate lyase activity on alginate oligo/polysaccharides such that it preferentially cleaves M-M in an Exo-fashion and yielding preferentially monosugars.

In other embodiments, more than one or more than two or more than three different lyases are used in a composition, wherein such multiple lyases may have different activities, such as but not limited to those activity characteristics as described in the preceding paragraph, and thus by choosing different lyases and ratios of these, a customized activity profile of a lyase mixture can be attained, as desired for various applications.

It follows that in some very useful embodiments of the invention, lyase(s) of the invention are used to degrade certain substrates of interest. In some embodiments the substrate which is degraded with the invention comprises a first segment consisting essentially of D-mannuronic acid residues (M), a second segment consisting essentially of L-guluronic acid residues (G), and a third segment consisting essentially of alternating D-mannuronic acid and L-guluronic acid residues (GM or MG).

In certain embodiments the substrate is an alginate oligosaccharide. In other embodiments the substrate is an alginate polysaccharide, which may or may not be derived from macroalgae. In other embodiments, the substrate comprises a mixture of alginate oligosaccharide and alginate polysaccharide. The invention provides useful in particular embodiments wherein the substrate comprises polysaccharide derived from macroalgae from the genera of Microcystis, Ascophyllum, Laminaria, Ecklonia or Sargassum.

The invention is herein below described with certain illustrative non-limiting examples.

### Example 1

This example demonstrates how putative alginate lyase encoding genes were identified by sequence similarity analysis using the NCBI BLAST program (non-redundant protein sequences database). Sequence Alignments were performed using the EBI ClustalW2-Multiple Sequence Alignment tool (http://www.ebi.ac.uk/). Molecular weight (MW) and isoelectric points (pI) were computed using the Compute pI/Mw tool (ExPASy). Protein sequence analysis was performed using InterPro (EMBL-EBI) and SMART (EMBL) databases. Signal peptides were predicted using the SignalP-4.0 Server (CBS).

The alyRm1, 2 and 4 genes were identified through sequence similarities with previously annotated alginate lyase genes (>40% aa identity). The alyRm3 gene was detected through non-obvious similarities following psi-BLAST where 23% aa identity was found with about 300 nt gene fragment encoding a partial alginate lyase protein from Yersinia pestis (WP_002427804.1) containing a heparinase II/III-like domain.

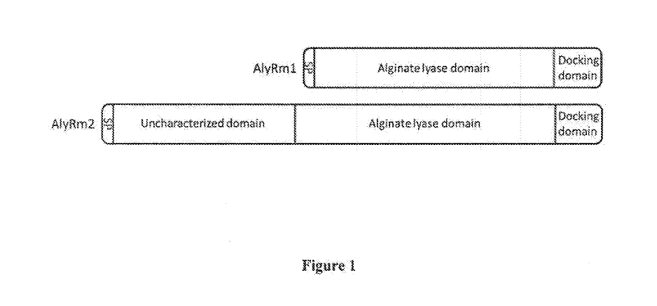

The alyRm1 was identified as a 1743 nt gene, encoding a 580 aa polypeptide with calculated MW of 63.683 and pI of 5.78. A putative signal sequence was predicted with cleavage site after Ala-19. The alyRm2 gene encodes a 2901 nt gene, encoding a 966 aa polypeptide with calculated MW of 107.238 and pI of 4.74. Both genes encode pectin lyase fold domains that contain parallel beta-helix repeats [20] often found in polysaccharide degrading enzymes. Based on their deduced aa sequences, both enzymes belong to family 6 of polysaccharide lyases. C-terminal domains of about 90 nt (10 kDa) are found in both genes. These domains show significant alignments with C-terminal modules predicted to mediate cell-attachment in members of the Bacteroides phylum [21]. A similar C-terminal region has been identified in a previously identified family 6 polysaccharide lyase from the thermophile Melioribacter roseus P3M (GenBank: AFN74606.1). The alyRm2 gene also contains a large N-terminal domain of about 1155 nt (43 kDa) of unknown function which contains no known motifs. Based on the above information the two R. marinus alginate lyases may be divided into the following regions: AlyRm1; signal peptide (aa 1-19), alginate lyase (aa 20-490) and C− terminal sorting/docking domain (aa 491-580) and AlyRm2; uncharacterized N-terminal domain, (aa 1-385), alginate lyase (aa 386-874) and C-terminal sorting/docking domain (aa 875-966) (see FIG. 1).

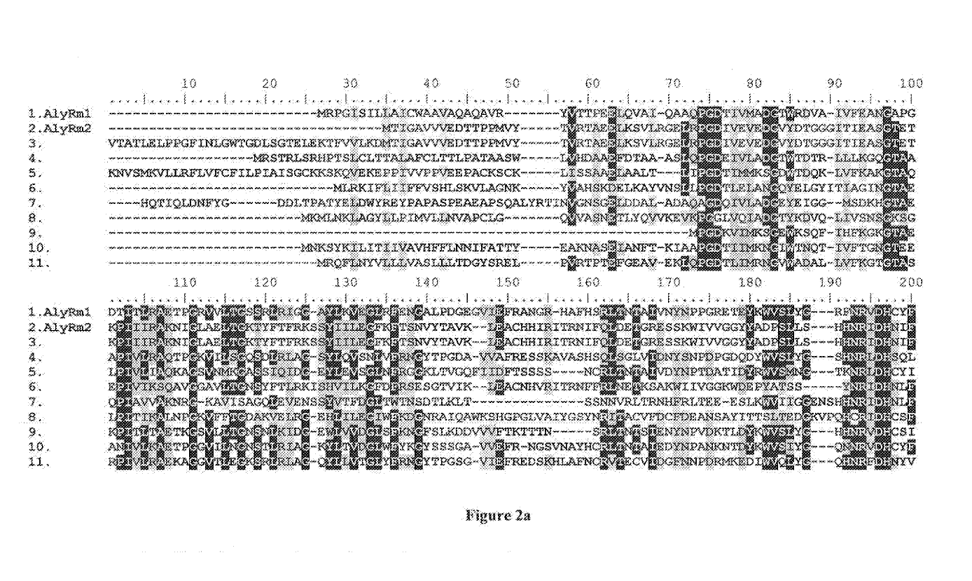

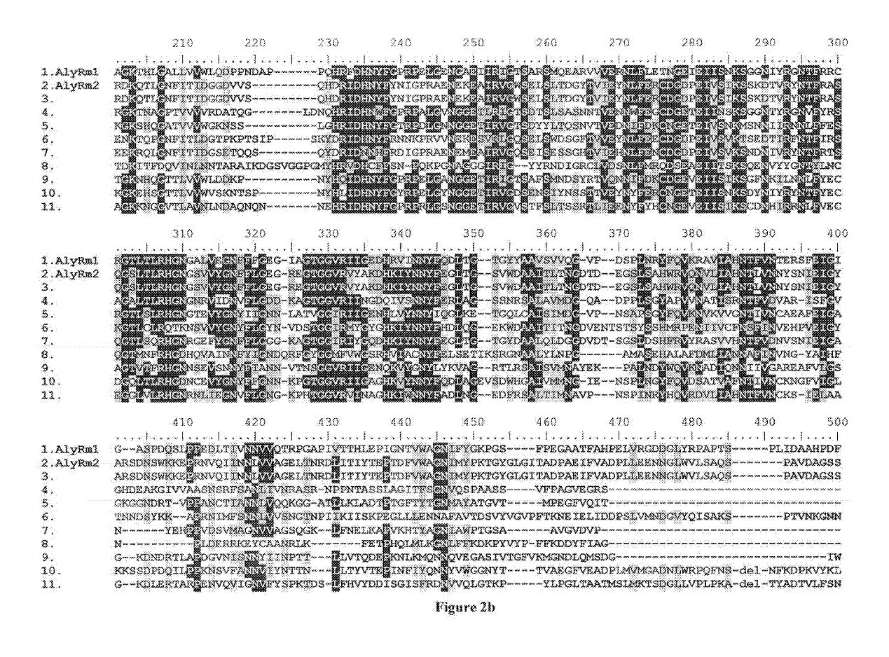

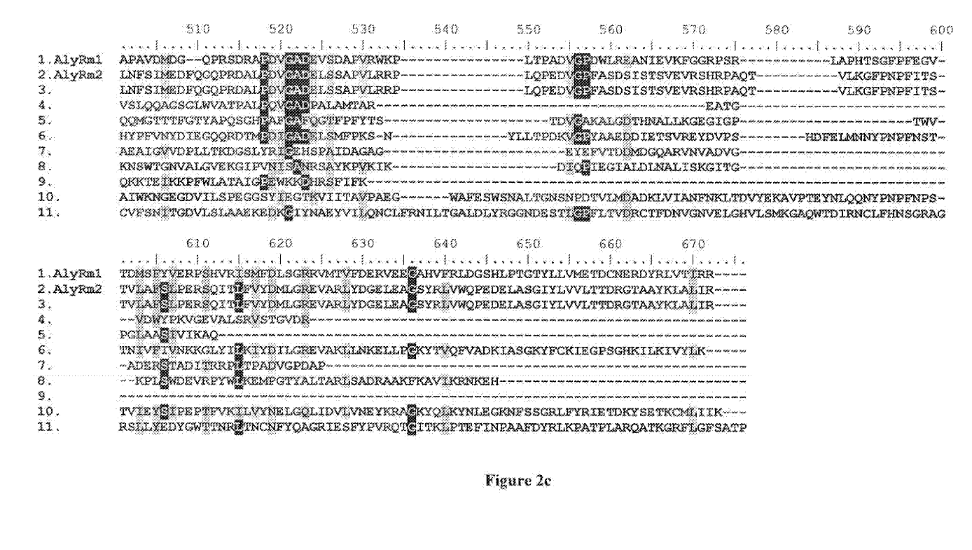

The defined alginate lyase domains of AlyRm1 (aa 20-490) and AlyRm2 (aa 386-874) shared 36% aa identity. AlyRm1 showed 92% aa sequence identity (lyase domain showed 93% aa identity) with annotated polyM lyase of R. marinus strain DSM 4252 (GenBank: ACY48055.1) and the lyase domain showed 49% aa identity with a polyM lyase of M. roseus PM3 (GenBank: AFN74598.1). AlyRm2 was identical with a hypothetical protein from R. marinus DSM 4252 (GenBank: ACY48275.1) and the lyase domain showed 46% aa identity with a lyase precursor from M. roseus (GenBank: AFN74606.1). FIG. 2 shows sequence alignments of AlyRm1 and AlyRm2 to related sequences of highest identity closest relatives and all characterized enzymes in polysaccharide lyase families PL6. Sequences 4, 8, and 9 have been characterized. The N-terminal end of AlyRm2 was not included in the alignments. Insertions in sequences 10 and 11 (around position 490) were deleted for simplification (and marked as del in the figure). The sequences in FIG. 2 are the following:

1. AlyRm1 Rhodothermus marinus str. 378 with signal peptide sequence
2. AlyRm2 Rhodothermus marinus str. 378 without N-terminal sequence
3. gi|268316944|ref|YP_003290663.1| hypothetical protein Rmar_1386 [Rhodothermus marinus DSM 4252]
4. gi|379046722|gb|AFC88009.1| polyMG-specific alginate lyase [Stenotrophomonas maltophilia]
5. gi|515827351|ref|WP_017258104.1| hypothetical protein [Pedobacter arcticus]
6. gi|397690365|ref|YP_006527619.1| lyase precursor [Melioribacter roseus P3M-2]
7. gi|522162944|ref|WP_020674152.1| hypothetical protein [Amycolatopsis nigrescens]
8. gi|1002527|gb|AAC83384.1| chondroitinase B precursor [Pedobacter heparinus]
9. gi|216849|dbj|BAA01182.1| alginate lyase [Pseudomonas sp.]
10. gi|1397690357|ref|YP_006527611.1| poly(β-D-mannuronate) lyase [Melioribacter roseus P3M-2]
11. gi|518835313|ref|WP_019991221.1| hypothetical protein [Rudanella lutea]

The predicted ORF of alyRm3 was 2613 nt, encoding a 870 aa polypeptide, with calculated MW of 96.492 and pI 5.28. A signal sequence was predicted with cleavage site after Gln-17 and a heparinase II/III-like protein domain (aa 374-518) [22] was detected. The enzyme does not show high sequence similarity with previously described polysaccharide lyases and therefore cannot be assigned to a family. Highest sequence similarities were found with an annotated heparinase II/III family protein from R. marinus DSM 4252 (>99% aa identity, GenBank: ACY48059.1) and a heparinase II/III family protein from Rhodopirellula sp. SWK7 (39% aa identity, GenBank: ZP-23730810.1).

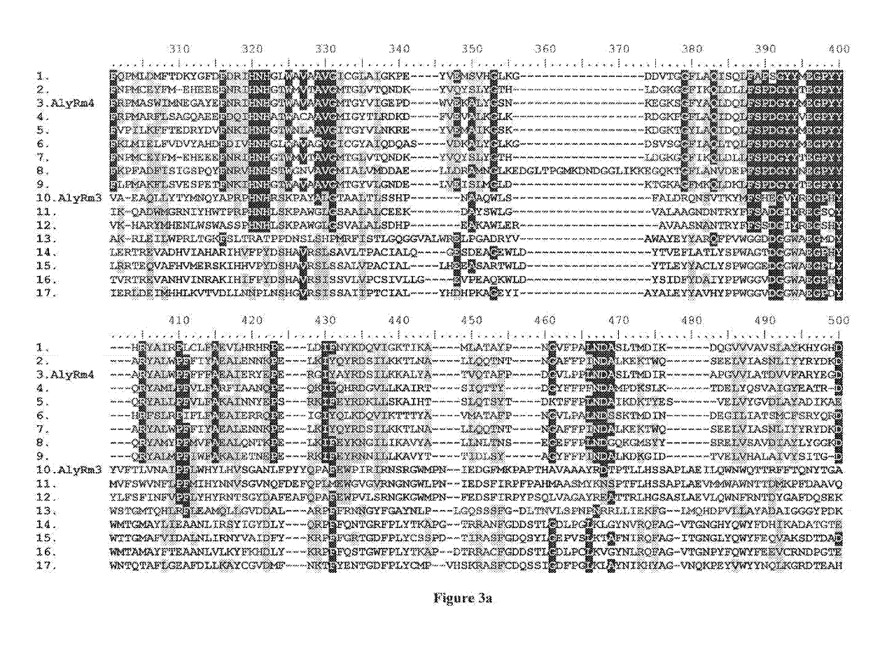

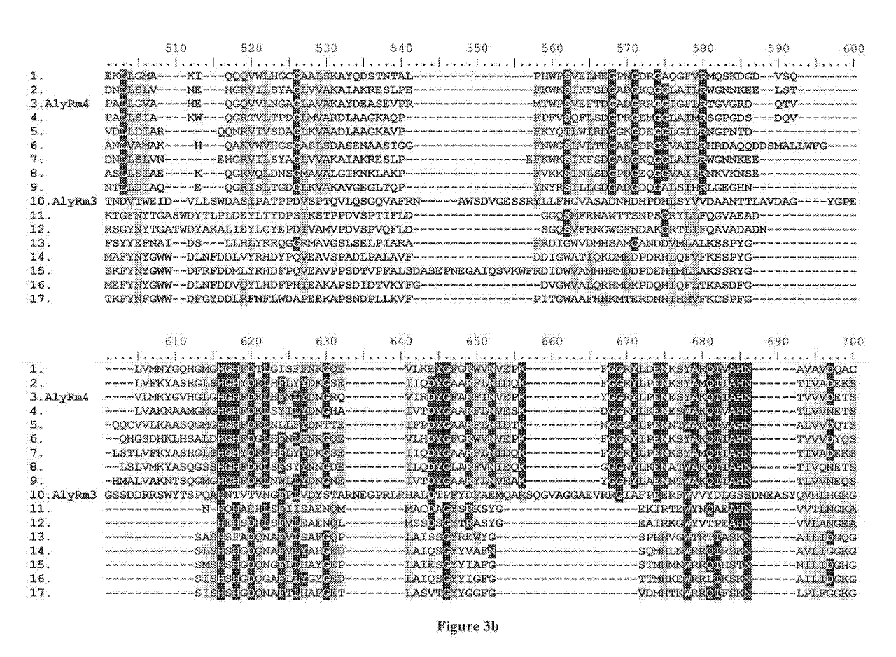

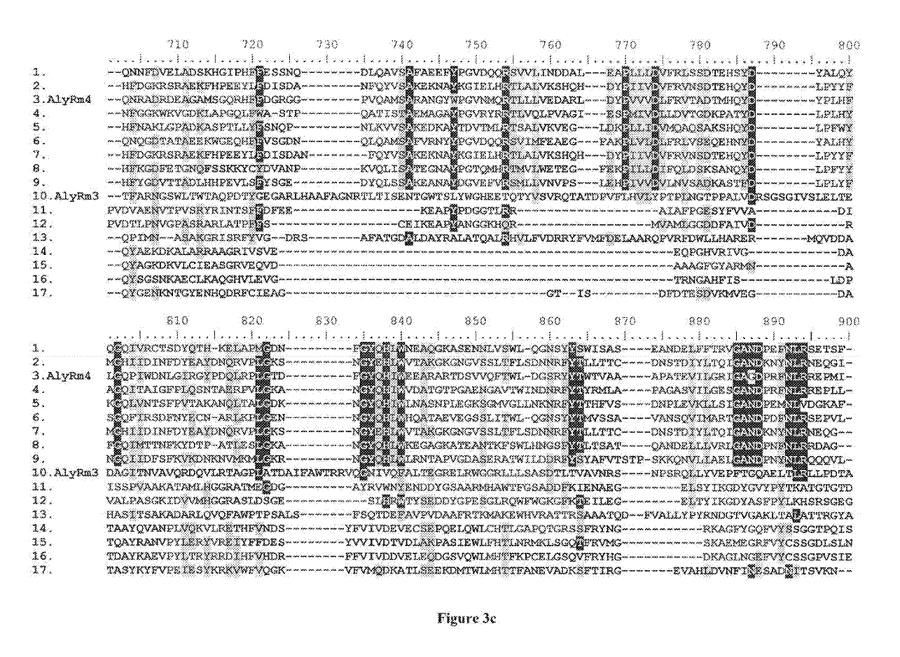

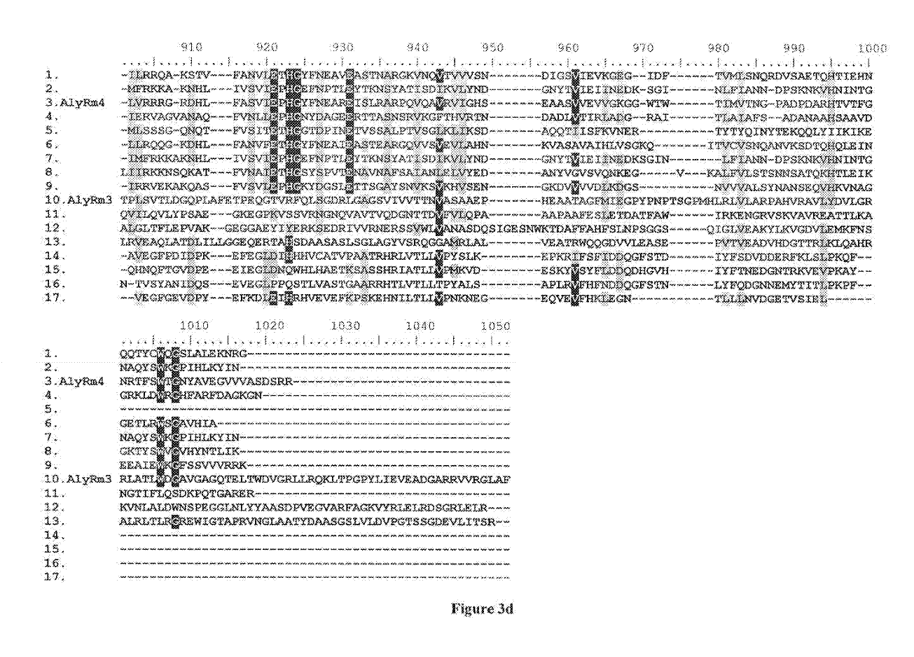

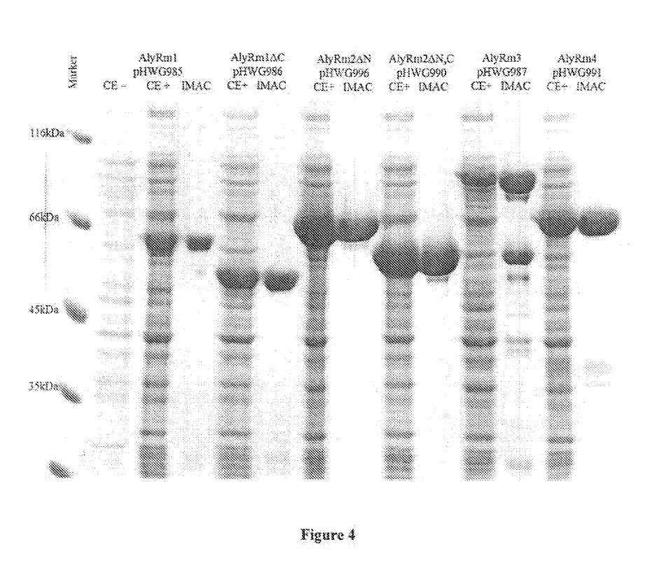

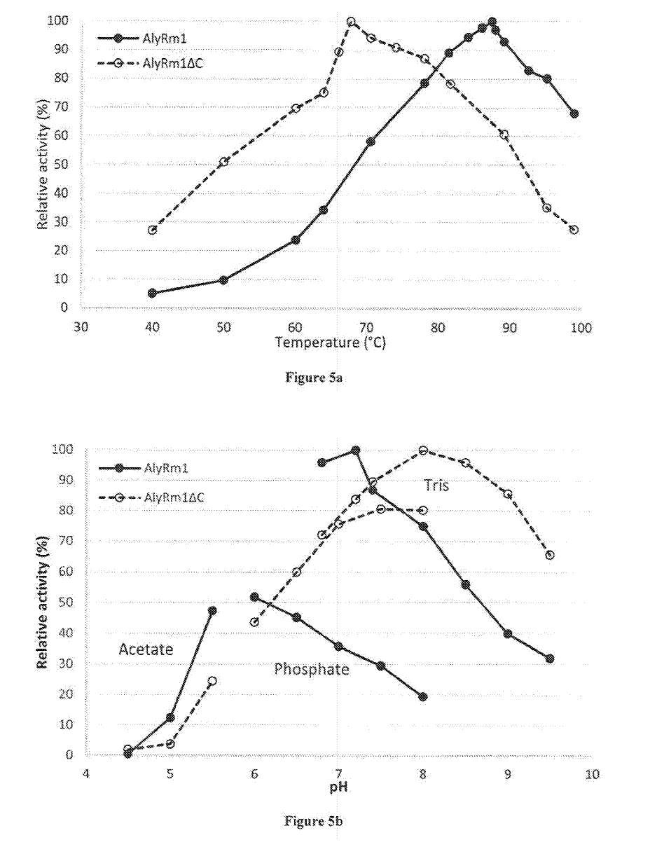

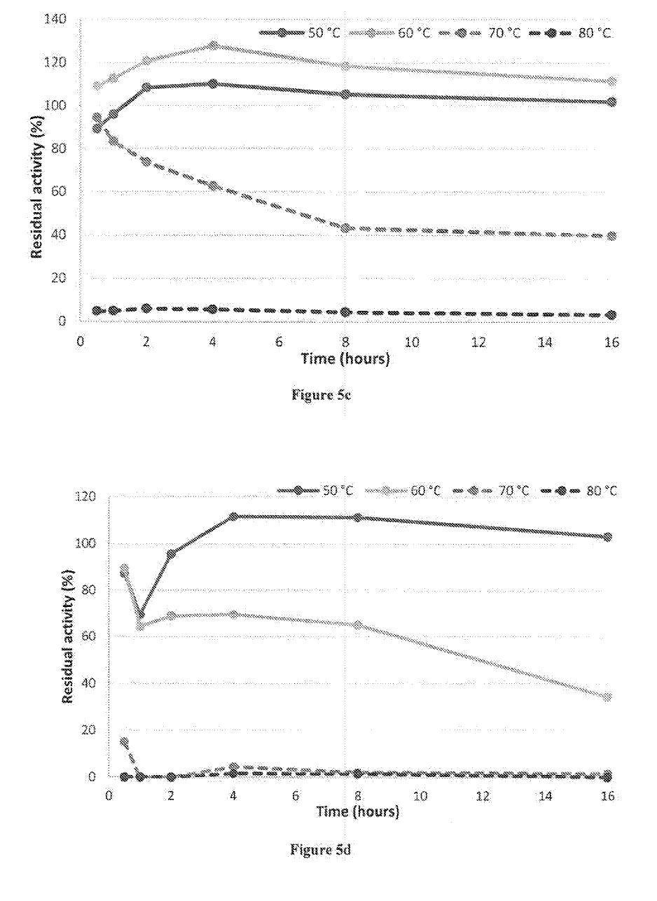

The gene encoding AlyRm4 consists of 2226 nt, which translate into a 742 aa polypeptide with MW of 83.561 and pI 6.11. A hydrophobic sequence was detected at the N-terminal end, indicating that the enzyme may be located in the periplasmic space. The enzyme contains both an alginate lyase domain (aa 26-309) and a heparinase II/III-like protein domain (aa 386-539). AlyRm4 belongs to family 17 of polysaccharide lyases and showed >99% aa identity with a heparinase II/III family protein from R. marinus DSM 4252 (GenBank: ACY48059.1) and 45% aa identity with a heparinase II/III family protein from M. roseus (GenBank: YP_006527616.1). FIG. 3 shows sequence alignments of AlyRm3 and AlyRm4 with similar sequences and characterized alginate lyases. The AlyRm sequences are aligned to related sequences of highest identity and all characterized enzymes in polysaccharide lyase families PL15 and PL17. Sequences 1-9 belong to family PL17 and sequences 13-17 belong to family PL15. Sequences 10-12 have not been assigned to a PL family. Sequences 4, 5, 8, 9, 13, 14 and 16 have been characterized as alginate lyases.

The sequences in FIG. 3 are the following:

1. gi|511825188|ref|WP_016403995.1| poly(β-D-mannuronate) lyase [Agarivorans albus]
2. gi|397690362|ref|YP_006527616.1| Heparinase II/III family protein [Melioribacter roseus P3M-2]
3. AlyRm4 Rhodothermus marinus str. 378 without N-terminal signal peptide
4. gi|342674030|gb|AEL31264.1| oligoalginate lyase [Sphingomonas sp. MJ3]
5. gi|217228794|gb|ACK10579.1|Pseudomonas sp. OS-ALG-9 Sequence 50 from U.S. Pat. No. 7,439,034
6. gi|511825189|ref|WP_016403996.1| poly(β-D-mannuronate) lyase [Agarivorans albus]
7. gi|397690362|ref|YP_006527616.1| Heparinase II/III family protein [Melioribacter roseus P3M-2]
8. gi|410825542|gb|AFV91542.1| alginate lyase 2B [Flavobacterium sp. S20]
9. gi|90022924|ref|YP_528751.1| alginate lyase [Saccharophagus degradans 2-40]
10. AlyRm3 Rhodothermus marinus str. 378 without N-terminal signal peptide
11. gi|496390129|ref|WP_009099119.1| Heparinase II/III family protein [Rhodopirellula sp. SWK7]
12. gi|495374567|ref|WP_008099279.1| Heparinase II/III-like protein [Verrucomicrobiae bacterium DG1235]
13. gi|60115421|dbj|BAD90006.1| alginate lyase [Sphingomonas sp. A1]
14. gi|15891901|ref|NP_357573.1| oligo alginate lyase [Agrobacterium fabrum str. C58]
15. gi|261407486|ref|YP_003243727.1| Heparinase II/III family protein [Paenibacillus sp. Y412MC10]
16. gi|9501763|dbj|BAB03319.1| oligo alginate lyase [Sphingomonas sp.]
17. gi|84376182|gb|EAP93067.1| hypothetical protein V12B01_24239 [Vibrio splendidus 12B01]

### Example 2

This example demonstrates the cloning of alginate lyase genes. The genes were amplified from the genome of R. marinus strain MAT378. The alyRm1 gene was amplified without the signal peptide sequence (without aa 1-17) and with and without the putative C-terminal cell-attachment domain (aa 491-580). They are designated AlyRm1 and AlyRm1ΔC, respectively. The alyRm2 gene was amplified without the N-terminal domain (aa 1-385) and with and without the putative C-terminal cell-attachment domain (aa 875-966). They are designated AlyRm2 and AlyRm2ΔC, respectively. The alyRm3 and alyRm4 genes were amplified without the predicted signal peptide sequences, AlyRM3 (without aa 1-17) and AlyRM4 (without aa 1-22) respectively. For heterologous expression in E. coli, all the alginate lyases genes were modified with an N-terminal hexa-histidine tag. Primers were designed as listed in Table 1 to amplify the coding regions of the respective genes and introducing the restriction sites BamHI or BglII at the 5′ ends and a HindIII site behind the stop codons. The amplifications were performed using standard PCR conditions and a proofreading polymerase, the fragments cut with the corresponding restriction enzymes and inserted into the L-rhamnose inducible expression vector pJOE5751. The vector contains a His6-eGFP fusion under control of the rhaP_{BAD} promoter. The single BamHI and HindIII restriction sites in the vector allowed the replacement of the eGFP by the alginate lyase genes and fusion to the His6-tag. All genes, the corresponding primers and the resulting expression vectors are listed in Table 1.

TABLE 1
Alginate lyase expression plasmids, alginate
lyase genes and primers for PCR amplification.

| Plasmid | Gene | Oligonucleotides used* |
|---|---|---|
| pHWG985 | alyRm1 | S8146: 5′-AAAAGATCTCAGG |
| | | CCGTCCGTTACGTG |
| | | S8147: 5′-AAAAAGCTTCAGC |
| | | GTCGTATGGTAACCAGT |
| pHWG986 | alyRm1\textcircled{x}C | S8146: 5′-AAAAGATCTCAGG |
| | | CCGTCCGTTACGTG |
| | | S8148: 5′-AAAAAGCTTCATC |
| | | CGAACTTCACTTCCGATGTTTG |
| pHWG996 | alyRm2\textcircled{x}N | S8321: 5′-AAAAAAGGATCCA |
| | | CGATCGGTGCGGTGGT |
| | | S8322: 5′-AAAAAAAAGCTTA |
| | | CCTGATCAGGGCAAGTT |
| pHWG990 | alyRm2\textcircled{x}NC | S8258: 5′-AAAGGATCCACGA |
| | | TCGGTGCGGTGGTG |
| | | S8225: 5′-AAAAAGCTTATAC |
| | | TTCAACGCTCGGTGCTGAT |
| pHWG987 | alyRm3 | S8149: 5′-AAAGGATCCCAGA |
| | | ACCCTTATGAGACTTACACG |
| | | S8150: 5′-AAAAAGCTTCTAA |
| | | AATGCCAGACCCCGGAC |
| pHWG991 | alyRm4 | S8259: 5′-AAAGGATCCCTGG |
| | | AAGTGCTCGCGCAGCC |
| | | S8227: 5′-AAAAAGCTTAACG |
| | | GCGGGAATCACTGGCAAC |

*Restriction sites for cloning are underlined.

### Example 3

This example demonstrates the expression of active enzymes in soluble form in E. coli, and purification to investigate their activity. An E. coli expression vector from Motejadded et al. [23] was used. E. coli JM109 carrying the respective recombinant plasmids were cultivated in LB medium (200 ml), containing 100 μg/ml ampicillin. For expression of the genes, cultures were grown at 37° C. till cell density reached OD₆₀₀ of 0.3, then induced by adding 0.1% rhamnose and further grown for 4 h at 30° C. The cells were harvested by centrifugation at 4500×g for 20 min at 4° C., washed, resuspended in 10 mM potassium phosphate buffer pH 6.5 and disrupted by passing them twice through a French press cell. After centrifugation (13,000×g for 15 min at 4° C.), the supernatants of the crude cell extracts and the cell pellets were analysed by SDS-PAGE.

The purifications of recombinant alginate lyase proteins were performed by immobilized metal affinity chromatography (IMAC). The supernatant of the respective crude cell extract, containing approximately 25 mg E. coli protein, was applied onto 2 ml Talon® metal affinity resin (Clontech) in a column using gravity flow. The resin was washed with 10 ml of washing buffer (50 mM potassium phosphate, 300 mM NaCl, 5 mM imidazole pH 7.0). Bound protein was eluted with 3 ml of elution buffer (50 mM potassium phosphate, 300 mM NaCl, 150 mM imidazole pH 7.0. (His)₆-alginase containing fractions were combined and applied onto an NAP10 column (GE Healthcare), equilibrated with 50 mM potassium phosphate, 300 mM NaCl pH 7.0 to remove imidazole and stored at 4° C.

Fractions containing purified samples were further analysed. Protein concentration was estimated using the method of Bradford [24] using Bradford reagent (Bio-Rad) and BSA standards for preparation of standard curves. Sodium dodecyl sulfate polyacrylamide gel electrophoresis (SDS-PAGE) was performed using the method of Laemmli [25] using standard gels and a broad-range protein standard (Fermentas). Gels were stained using Coomassie Brilliant Blue R-250 (Sigma). Rhamnose induction of the E. coli JM109, harbouring the respective plasmids, resulted in the production of high amounts of the recombinant proteins as judged by SDS-PAGE (FIG. 4). Compared to the non-induced crude extracts, a prominent protein band of the size of 63 kDa for AlyRm1, 53 kDa for AlyRm1 AC, 66 kDa for AlyRm2ΔN, 53 kDa for AlyRm2ΔNC, 97 kDa for AlyRm3 and 81 kDa for AlyRm4, respectively, indicated a tight regulation and high expression level. Recombinantly expressed alginate lyases were purified by IMAC to homogeneity as judged by SDS-PAGE (FIG. 4). The figure shows 10% SDS-PAGE of crude extracts (15 μg protein) of E. coli JM109 harbouring the respective plasmids for the alginate lyase genes and purified (His)₆-Alginate lyases (3 μg protein) after IMAC. CE− non-induced crude extract; CE+ crude cell extract from rhamnose-induced cells.

### Example 4

This example demonstrates the activity of the alginate lyases on alginate from Macrocystis pyrifera (Kelp) (low viscosity, sodium salt alginate obtained from Sigma). Samples (10 μl) were incubated for 10 min with 1% alginate in 50 mM buffer (90 μl) (final concentrations) at different temperatures. Then, 100 μl of 3,5-dinitrosalicylic acid (DNS) were added to each sample and heated at 100° C. for 5 min. A 150 μl of each sample were diluted with 150 μl of water and optical density (OD) measured at 546 nm. One unit (U) of enzyme activity corresponds to the release of 1 μmol of reducing sugar equivalents (expressed as glucose) per minute. For enzyme characterization, the following buffers were used; sodium acetate (pH 4.5-5.5), potassium phosphate (pH 6.0-8.0), and Tris (pH 7.2-9.5). Tris buffers were specifically set to work at the appropriate incubation temperatures.

All six recombinant enzymes degraded alginate and their characteristics are summarized in Table 2.

The two AlyRm1 variants displayed somewhat different characteristics. The AlyRm1 variant containing the C-terminal docking domain (AlyRm1) had a higher optimum temperature, was more heat stable and less salt tolerant than the variant lacking the domain (AlyRm1ΔC), see FIG. 5. Furthermore, some hindrance was detected when using phosphate buffer for measuring AlyRm1 activity. FIG. 5 shows characterization of alginate lyase AlyRm1 variants containing the C-terminal domain (AlyRm1) or lacking the C-terminal domain (AlyRm1ΔC). Unless otherwise indicated the enzymes were assayed at their optimum temperature and pH for 10 min. For assaying thermal stability, residual activity after incubation at 50, 60, 70 or 80° C. for up to 16 h was assayed at 60° C.

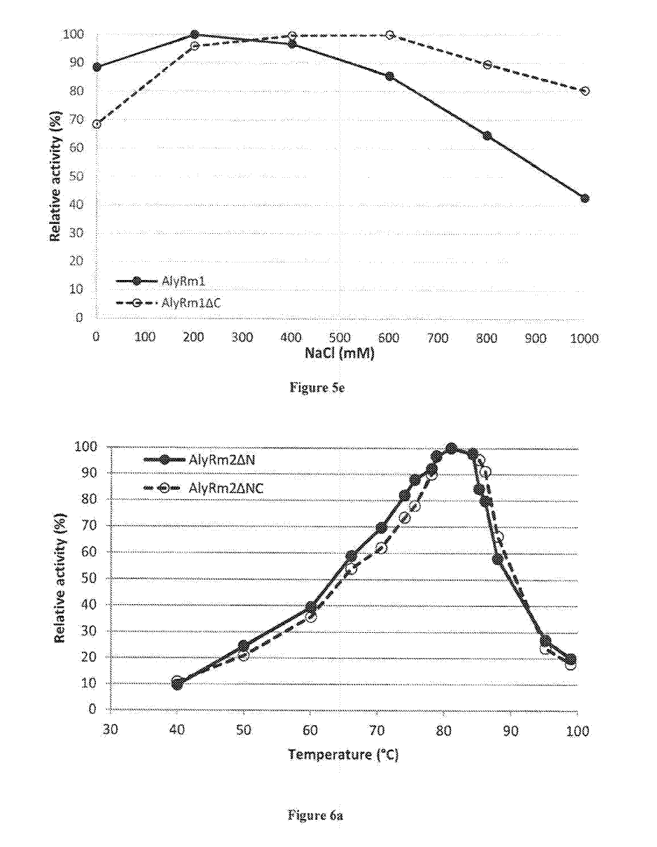

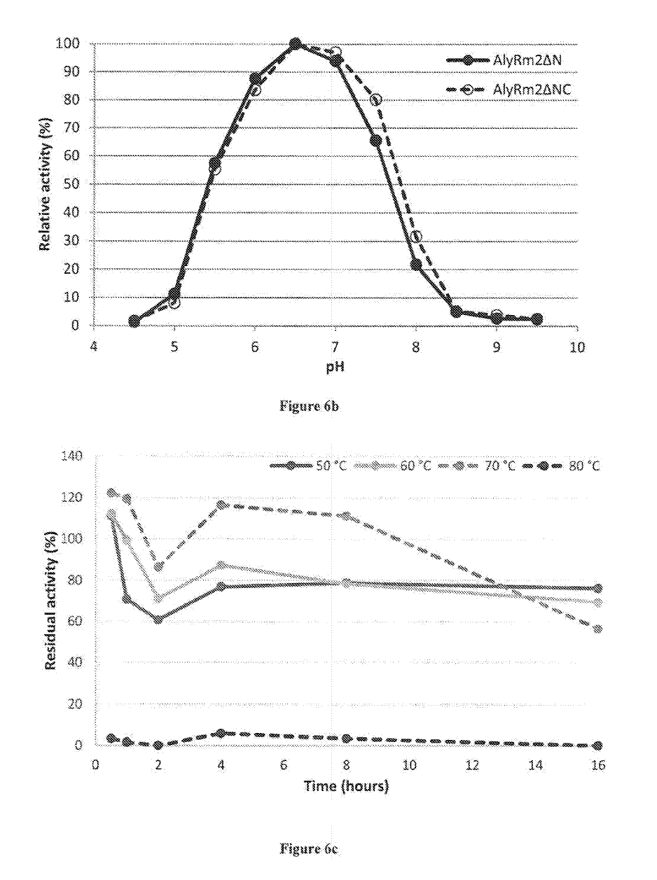

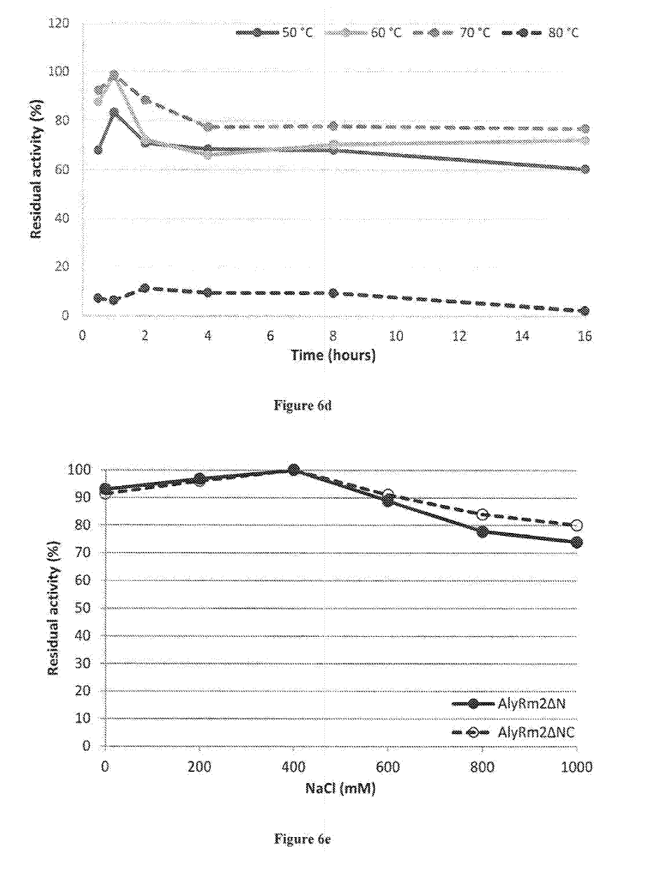

The two variants of AlyRm2 showed similar characteristics (FIG. 6), with temperature optimum around 81° C., pH optimum around 6.5, heat stability at 70° C. and they were not highly affected by variable salt concentration up to 1 M NaCl.

FIG. 6. Characterization of the alginate lyase AlyRm2 variants lacking the N-terminal domain (AlyRm2ΔN) or lacking both the N-terminal and C-terminal domains (AlyRm2ΔNC). Unless otherwise indicated, the enzymes were assayed at their optimum temperature and pH for 10 min. For assaying thermal stability, residual activity after incubation at 50, 60, 70 or 80° C. for up to 16 h was assayed at 60° C.

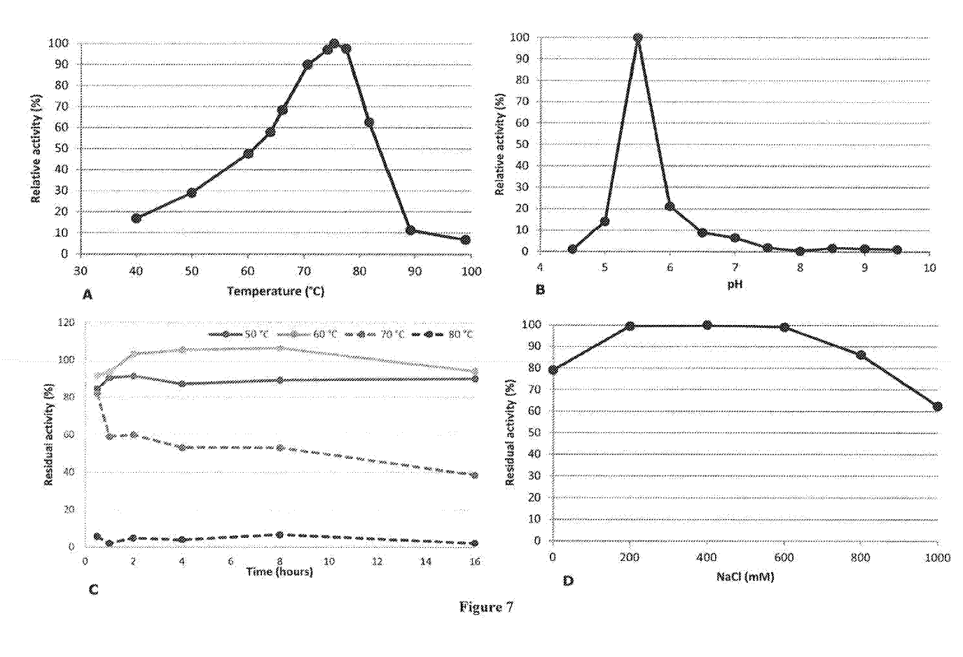

AlyRm3 was most active at around 75° C. and had a very narrow pH range around 5.5. The enzyme half-life was estimated around 8 h at 70° C. The enzyme was relatively stable at variable concentrations of NaCl (FIG. 7). The figure shows characterization of alginate lyase AlyRm3. A) optimum temperature, B) optimum pH, C) thermal stability, D) optimum salinity. Unless otherwise indicated the enzymes were assayed at their optimum temperature and pH for 10 min. For assaying thermal stability, residual activity after incubation at 50, 60, 70 or 80° C. for up to 16 h was assayed at 60° C.

The optimum temperature of AlyRm4 was 81° C. and the enzyme was heat stable at 70° C. for at least 16 h. The optimum pH was 6.5 and the enzyme was relatively stable at NaCl concentrations up to 1 M (FIG. 8). The figure shows characterization of alginate lyase AlyRm4. A) optimum temperature, B) optimum pH, C) thermal stability, D) optimum salinity. Unless otherwise indicated the enzymes were assayed at their optimum temperature and pH for 10 min. For assaying thermal stability, residual activity after incubation at 50, 60, 70 or 80° C. for up to 16 h was assayed at 60° C.

### Example 5

This example demonstrates the degradation of alginate following incubation with the recombinant enzymes and assayed using thin layer chromatography (TLC) at different reaction times. Reaction products were visualized on the TLC plate by developing with the solvent mixture n-butanol/acetic acid/water (2:1:1, by volume) and visualized using 2.5% sulfuric acid solution in 47.5% ethanol, followed by heating the TLC plate at 100° C. for 10 min.

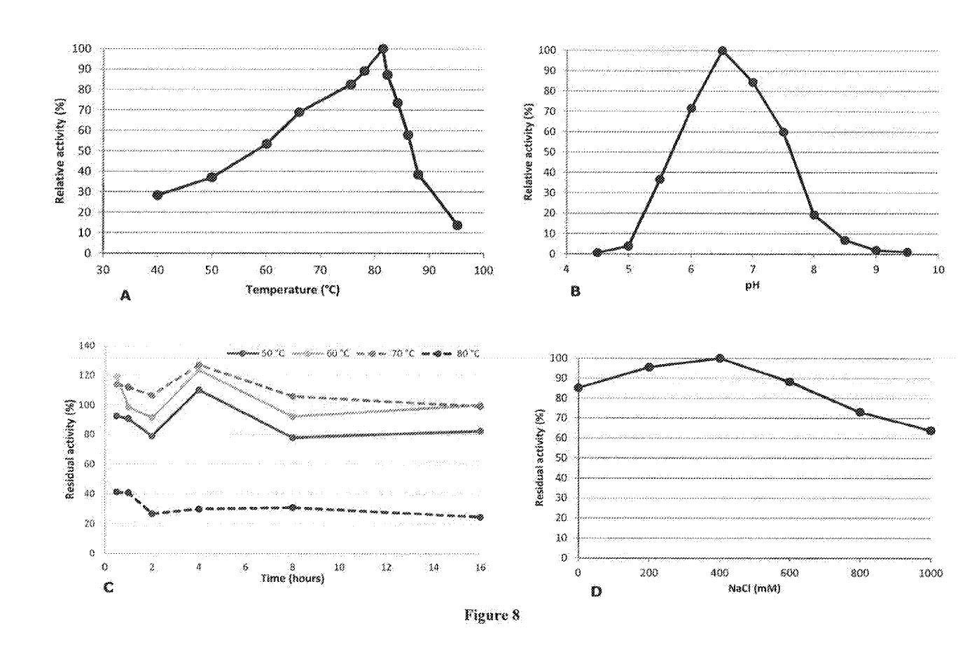

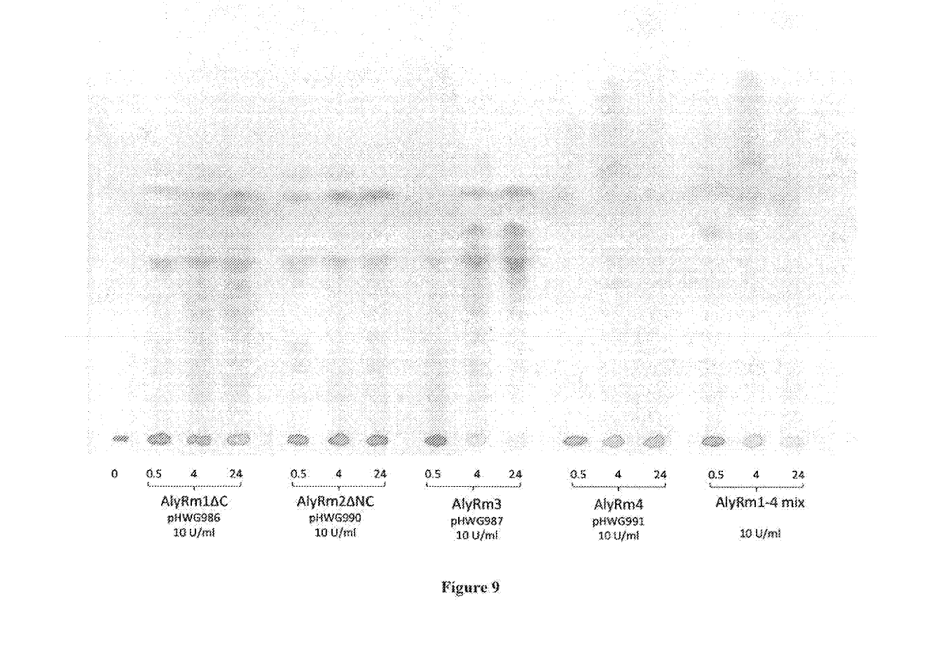

The AlyRm1, AlyRm2 and AlyRm3 lyases produced different patterns of unidentified oligosaccharides, whereas AlyRm4 and a mix of all four enzymes seemed to produce mostly (unsaturated) mono-uronates, not detected by TLC (FIG. 9).

FIG. 9 shows thin layer chromatography (TLC) showing alginate degradation by the thermostable alginate lyase enzymes. The substrate used was 1% sodium alginate. Incubation was at 60° C. and pH 7.0 for 0.5, 4 and 24 h (X-axis).

None of the alginate lyase enzymes showed any activity on chondroitin sulfate.

### Example 6

This example demonstrates the degradation pattern of the four thermophilic alginate lyase enzymes when analyzed with TLC, MALDI-TOF-MS, HPAEC-PAD and 1D/2D ¹H NMR. The methods used for the analysis have been previously described, i.e. in Hreggvidsson et al. [26] and in Jonsson [27]. Enzyme incubations were done as follows: Mixing 80 μl of 12.5 mg/ml of alginate (from Sigma), or G-block oligosaccharides, or M-block oligosaccharides (from Elicityl) into 10 μl of 0.5 M phosphate buffer pH 7 and 10 μl of 0.5 U/ml enzyme solution, followed by incubation at 65° C. for the appropriate time.

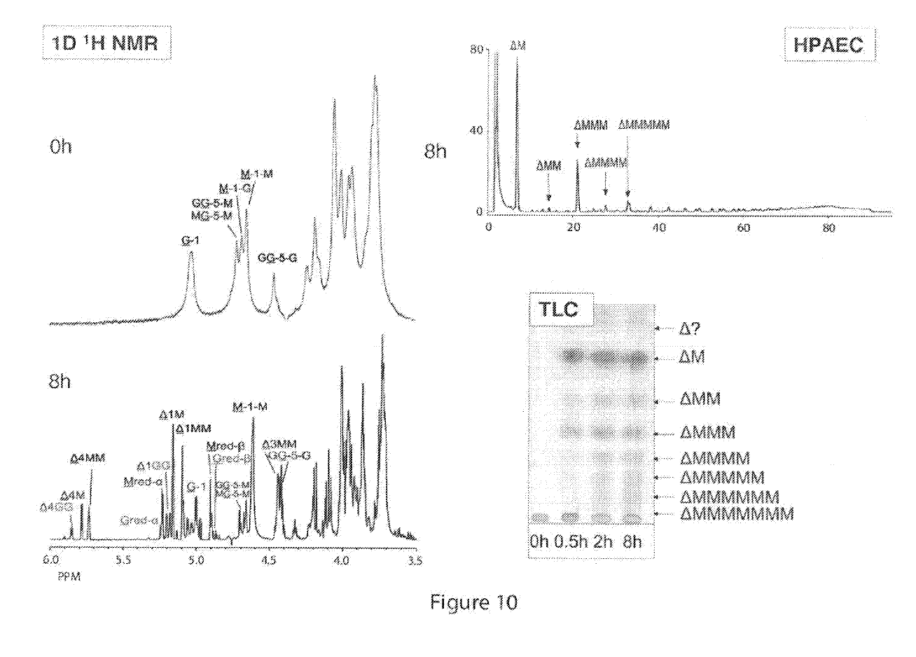

Degradation pattern of alginate, G-block and M-block by the thermostable alginate lyase, AlyRm1 is shown in FIGS. 10, 11 and 12, respectively.

FIG. 10 shows degradation pattern after 8 h incubation of alginate with the thermostable alginate lyase, AlyRm1, as analyzed with 1D ¹H NMR, HPAEC-PAD and TLC. Δ=4,5-unsaturated uronic acid; ΔM=disaccharide of terminal 4,5-unsaturated uronic acid, (1→4)-linked to D-mannuronic acid, and so on.

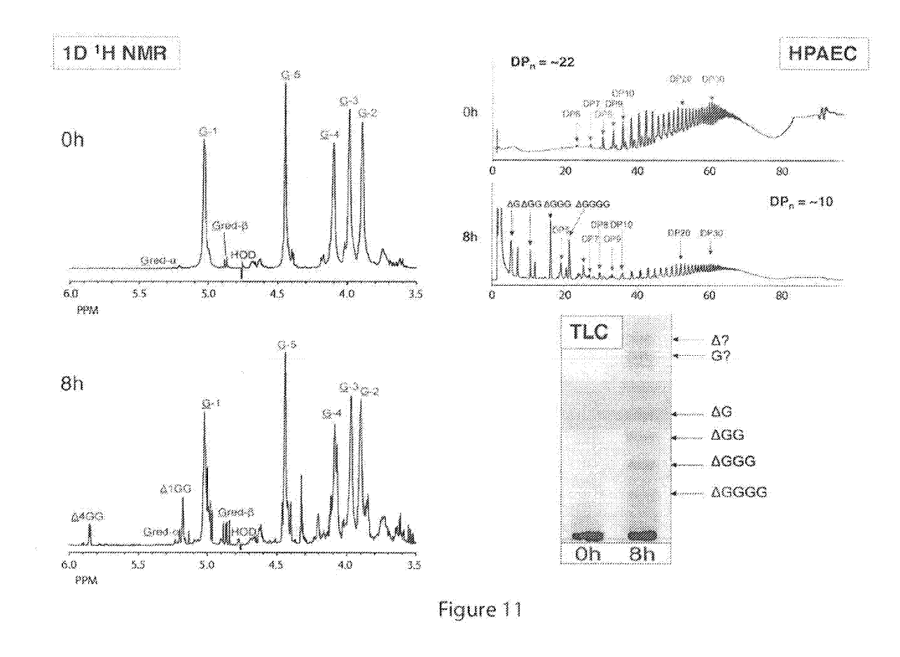

FIG. 11 shows degradation pattern after 8 h incubation of a G-block with the thermostable alginate lyase, AlyRm1, as analyzed with 1D ¹H NMR, HPAEC-PAD and TLC. Δ=4,5-unsaturated uronic acid; ΔG=disaccharide of terminal 4,5-unsaturated uronic acid, (1→4)-linked to L-guluronic acid, and so on.

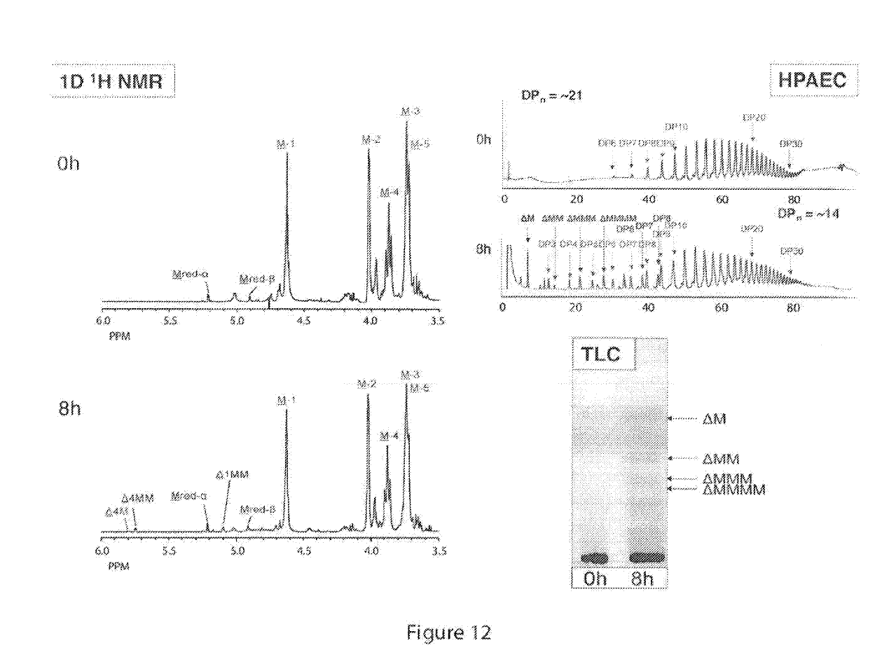

FIG. 12 shows degradation pattern after 8 h incubation of an M-block with the thermostable alginate lyase, AlyRm1, as analyzed with 1D ¹H NMR, HPAEC-PAD and TLC. Δ=4,5-unsaturated uronic acid; ΔM=disaccharide of terminal 4,5-unsaturated uronic acid, (1→4)-linked to D-mannuronic acid, and so on.

The data in FIGS. 10, 11 and 12 show that AlyRm1 is an exotype and endotype alginate lyase with major activity in cleaving M-G bonds but not G-M bonds and with only minor activity in cleaving G-G bonds and M-M bonds: ( . . . M↓GMMM↓GGGGGGGGM↓MMMMMM↓GM↓GM↓GM↓GM↓MMMMMMM . . . )

The same enzyme lacking the C-terminal domain (AlyRm1ΔC), however has major activity in cleaving G-G, M-G and/or G-M bonds but with minor activity on M-M bonds (results not shown): ( . . . M↓GMMM↓G↓GG↓GGG↓G↓G↓MMMMMMM↓GM↓GM↓GM↓GM↓MMMM).

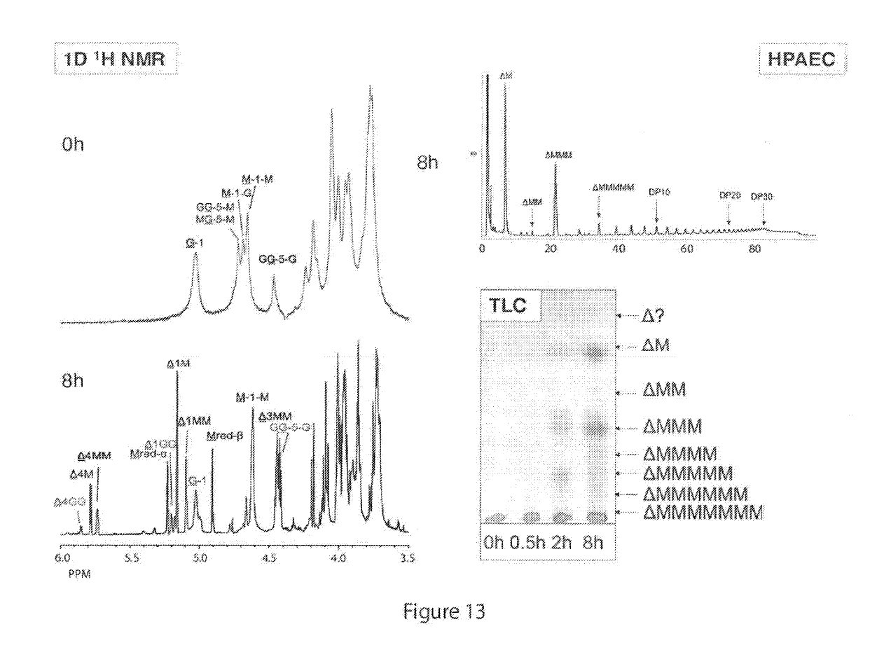

Degradation pattern of alginate, G-block and M-block by the thermostable alginate lyase, AlyRm2ΔNC is shown in FIGS. 13, 14 and 15.

FIG. 13 shows degradation pattern after 8 h incubation of alginate with the thermostable alginate lyase, AlyRm2ΔNC, as analyzed with 1D ¹H NMR, HPAEC-PAD and TLC. Δ=4,5-unsaturated uronic acid; ΔM=disaccharide of terminal 4,5-unsaturated uronic acid, (1→4)-linked to D-mannuronic acid, and so on.

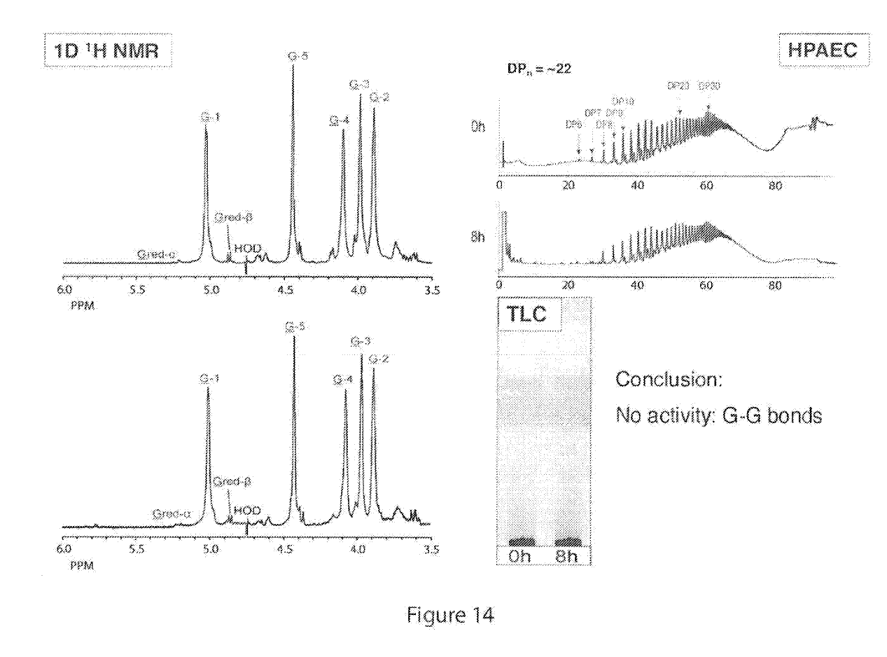

FIG. 14 shows degradation pattern after 8 h incubation of a G-block with the thermostable alginate lyase, AlyRm2ΔNC, as analyzed with 1D ¹H NMR, HPAEC-PAD and TLC.

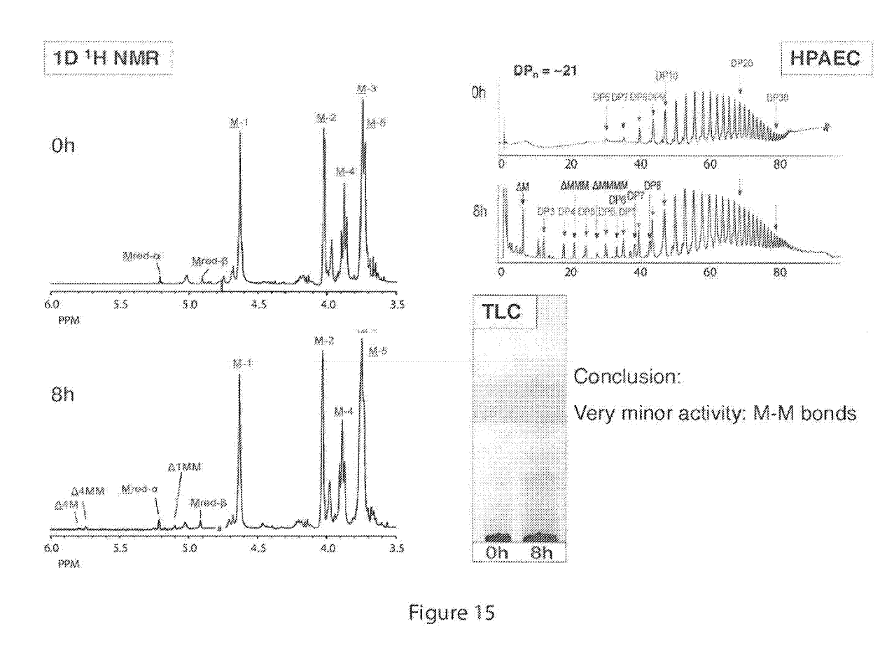

FIG. 15 shows degradation pattern after 8 h incubation of an M-block with the thermostable alginate lyase, AlyRm2ΔNC, as analyzed with 1D ¹H NMR, HPAEC-PAD and TLC.

The data in FIGS. 13, 14 and 15 show that AlyRm2ΔNC is an endotype alginate lyase with major activity in cleaving M-G bonds but not G-M bonds and with only minor activity in cleaving M-M and no activity in cleaving G-G bonds: ( . . . M↓GMMM↓GGGGGGGGM↓MMMMMM↓GM↓GM↓GM↓GM↓MMMMMMM . . . ).

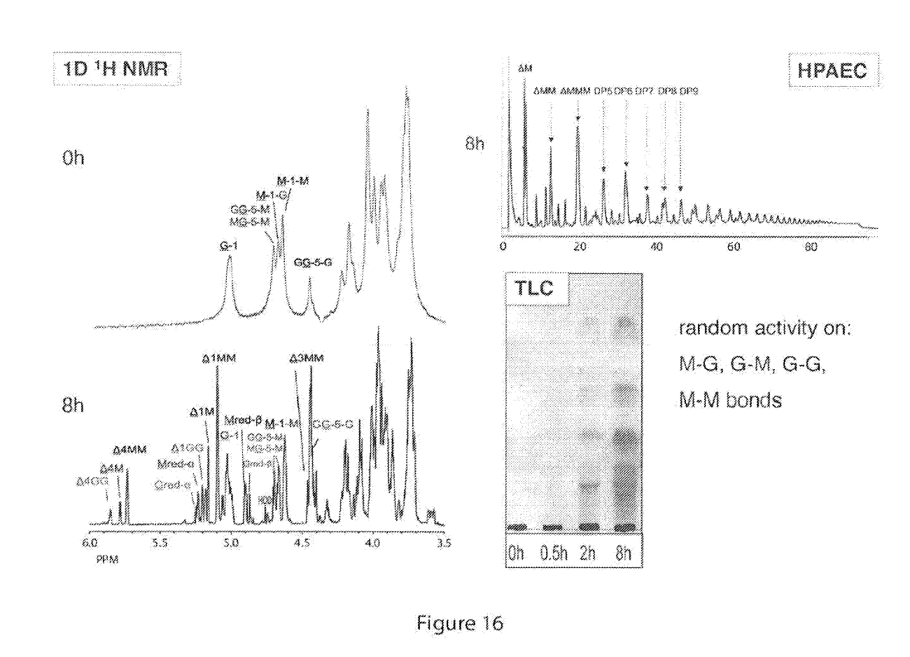

The degradation pattern of alginate, G-block and M-block by the thermostable alginate lyase, AlyRm3 is shown in FIGS. 16, 17 and 18.

FIG. 16 shows degradation pattern after 8 h incubation of alginate with the thermostable alginate lyase, AlyRm3, as analyzed with 1D ¹H NMR, HPAEC-PAD and TLC.

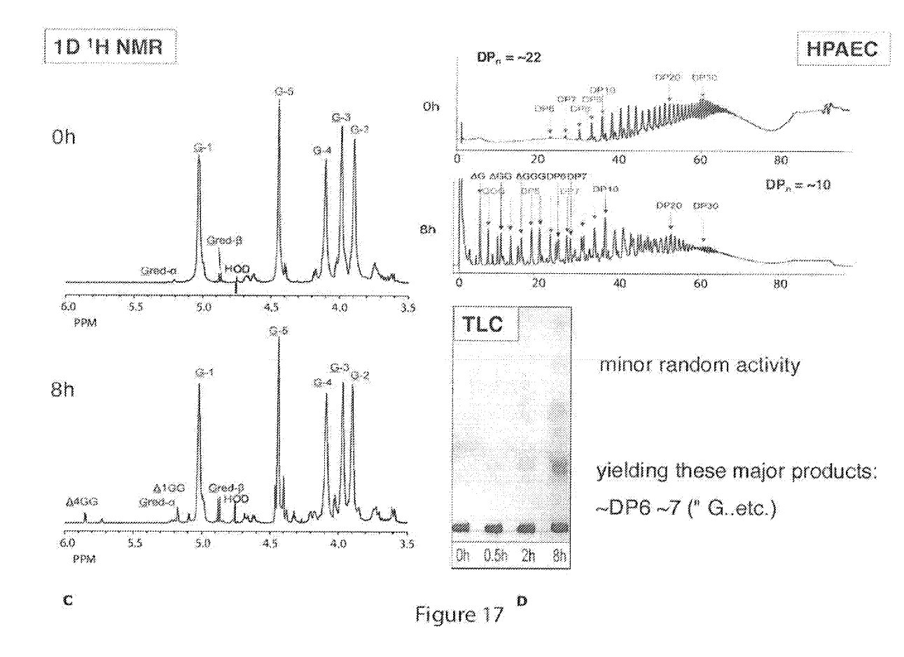

FIG. 17 shows degradation pattern after 8 h incubation of a G-block with the thermostable alginate lyase, AlyRm3, as analyzed with 1D ¹H NMR, HPAEC-PAD and TLC.

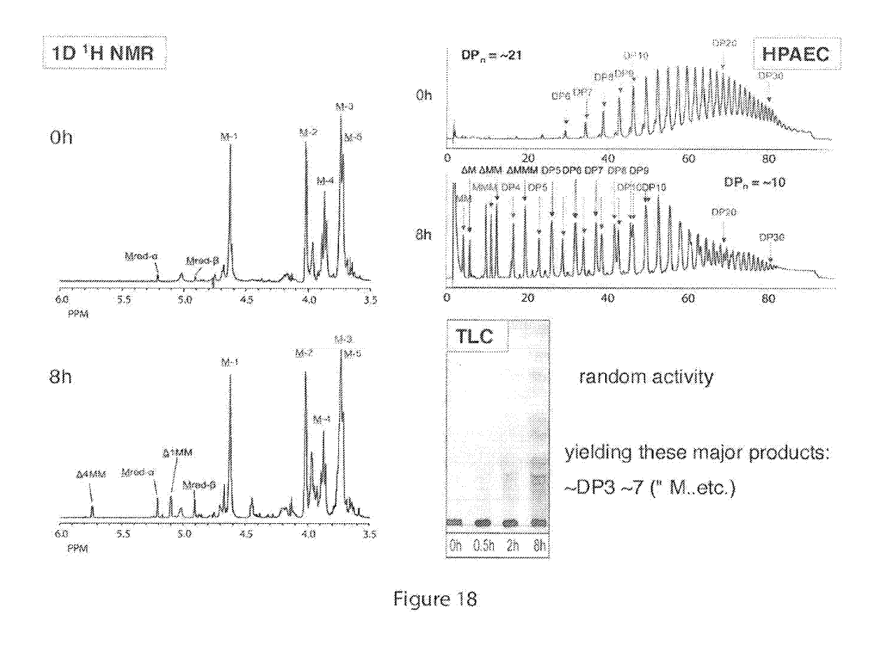

FIG. 18 shows degradation pattern after 8 h incubation of an M-block with the thermostable alginate lyase, AlyRm3, as analyzed with 1D ¹H NMR, HPAEC-PAD and TLC.

The data in FIGS. 16, 17 and 18 show that AlyRm3 is an endotype alginate lyase that cleaves all of the bonds, M-G, G-M, G-G and M-M at random but apparently with some preference for the M-M bonds: ( . . . M↓GMMM↓GGGGG↓GGGGM↓MMMM↓MM↓GMG↓MGM↓GM↓MMMMMMM . . . ).

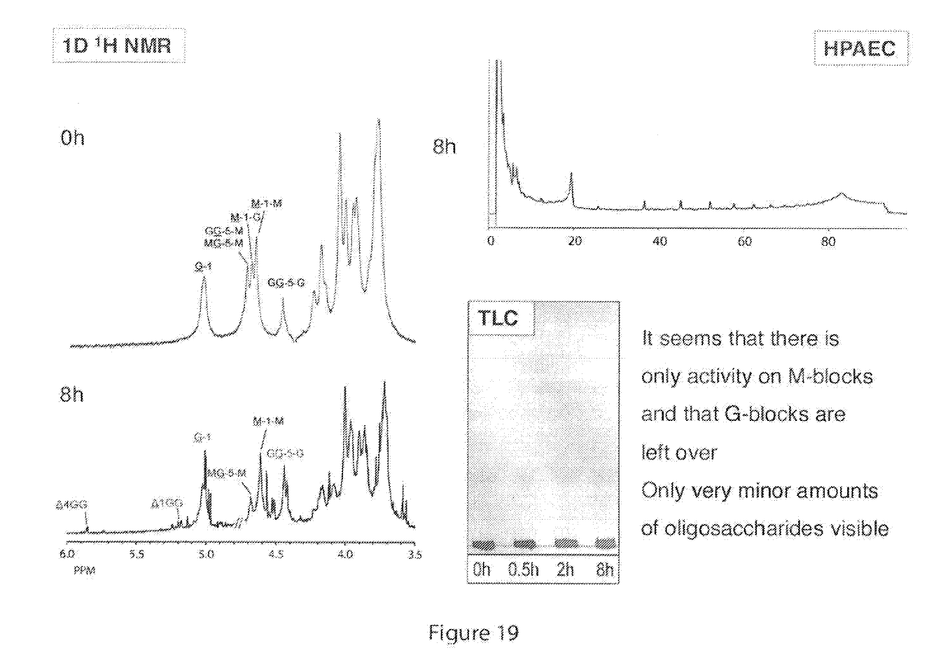

The degradation pattern of alginate, G-block and M-block by the thermostable alginate lyase, AlyRm4 is shown in FIGS. 19, 20 and 21.

FIG. 19 shows degradation pattern after 8 h incubation of alginate with the thermostable alginate lyase, AlyRm4, as analyzed with 1D ¹H NMR, HPAEC-PAD and TLC.

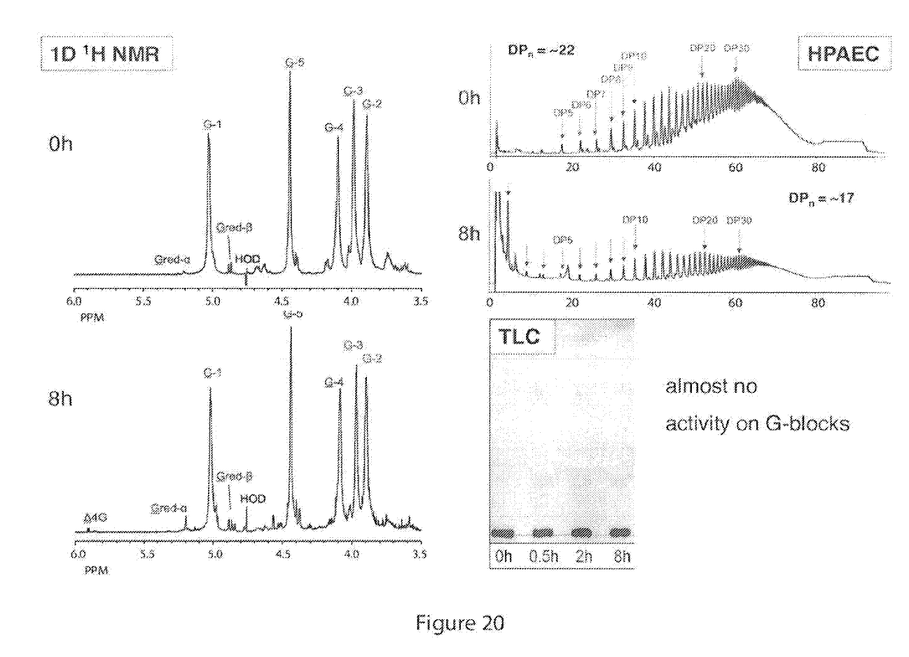

FIG. 20 shows degradation pattern after 8 h incubation of a G-block with the thermostable alginate lyase, AlyRm4, as analyzed with 1D ¹H NMR, HPAEC-PAD and TLC.

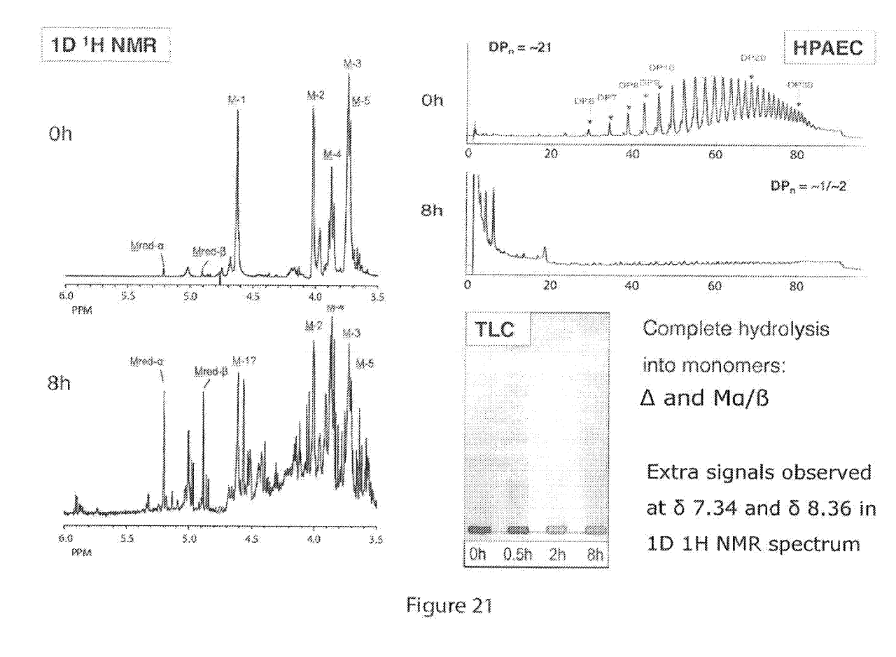

FIG. 21 shows degradation pattern after 8 h incubation of an M-block with the thermostable alginate lyase, AlyRm4, as analyzed with 1D ¹H NMR, HPAEC-PAD and TLC.

The data in FIGS. 19, 20 and 21 shows that AlyRm4 is an exotype alginate lyase that has major activity in cleaving M-M bonds, resulting in complete degradation of all M-blocks into monomers of Δ and Mα/β. The enzyme shows only minor activity in cleaving G-G bonds: (M↓M↓M↓M↓M↓M↓G↓M↓G↓M↓G↓M↓G↓M↓M↓M↓M↓M↓M↓M↓GGGGGGGGG . . . ).

The main characteristics of the thermostable alginate lyases from R. marinus are summarized in Table 2.

TABLE 2
Main characteristics of the thermostable alginate lyases and their variants from R. marinus.

| Enzyme | AlyRm1 | AlyRm1ΔC | AlyRm2ΔN | AlyRm2ΔNC | AlyRm3 | AlyRm4 |
|---|---|---|---|---|---|---|
| T opt (° C.) | 87 | 68 | 81 | 81 | 75 | 81 |
| T stability | 5 h at 70° C. | 12 h at 60° C. | 16 h at 70° C. | >16 h at 70° C. | 8 h at 70° C. | >16 h at 70° C. |
| (enzyme half-life)¹ | >16 h at 60° C. | | >16 h at 60° C. | | >16 h at 60° C. | |
| pH opt | 7.2 | 8.0 | 6.5 | 6.5 | 5.5 | 6.5 |
| NaCl opt.² (mM) | 0-600 | 200-1000 | 0-800 | 0-800 | 0-800 | 0-600 |
| Enzyme type | Endo & Exo | Endo & Exo | nd | Endo | Endo | Exo |
| Major activity | M-G | M-G, G-G | nd | M-G | All | M-M |
| Minor activity | G-G, M-M | M-M | nd | M-M | | G-G |

¹Enzyme half-life estimated as 50% residual activity following incubation at 50, 60, 70 or 80° C. for up to 16 h. All enzymes were fully stable at 50° C. but none of them was stable following 30 min. incubation at 80° C. Assay performed at 60° C. (optimum pH).
²≧80% relative activity.
Nd, not determined

### Example 7

This example demonstrates the extent of degradation of alginate into (unsaturated) mono-uronates by the four thermophilic alginate lyase enzymes of the invention. A 4 ml reaction mixture composed of 1% alginate (10 mg/ml) in 50 mM acetate buffer (pH 5.5) and containing 0.6 U/ml of each enzyme (AlyRm1, AlyRm2, AlyRm3, AlyRm4) was incubated at 55° C. with shaking at 150 rpm for 24 h.

To a 0.8 ml sample of the above reaction mixture (or dilutions thereof) was then added the following: 25 μl of 25 mM HIO₄ in 0.125M H₂SO₄ and kept at room temperature for 20 min.

Then 0.5 ml of 2% Sodium arsenite in 0.5M HCl and 2 ml of 0.3% TBA solution (2-Thiobarbituric Acid, pH 2) were added, stirred and incubated at 100° C. for 10 min. Then cooled and absorbance was measured at 548 nm. (The method is based upon the formation of β-formylpyruvate from periodate oxidation, where 0.01 μmol β-formylpyruvate gives optical density reading of 0.29 at 548 nm [ε=2.9×10⁴ M⁻¹ cm⁻¹]. The standard curve made with 2-deoxy glucose (10 mg/ml) was linear up to 0.08 mg of 2-deoxy glucose in the above assay. Incubation with different combinations of the thermophilic alginate lyases were also in all cases linear up to 0.08 mg of alginate and therefore the extent of degradation into unsaturated monomers could be determined. The different enzyme combinations gave results as shown in Table 3.

TABLE 3
Degradation of alginate into unsaturated monomers by thermophilic
alginate lyase enzymes in different compositions.

| | Degree (%) of conversion into unsaturated monomers | | | |
|---|---|---|---|---|
| | Enzyme in reaction | | | |
| | | | AlyRm3, | AlyRm1, AlyRm2, |
| Substrate | AlyRm3, | AlyRm4 | AlyRm4 | AlyRm3, AlyRm4 |
| Alginate | 53% | 68% | 99% | 85% |
| G-block | 42% | 30% | | |
| M-block | 39% | 85% | | |

The data presented in this example shows that different degree of conversion into unsaturated monomers can be obtained from around 50% to around 100%, by using different alginate lyases of the invention or mixtures thereof.

### REFERENCES

1. Gao, K. and K. R. McKinley, Use of Macroalgae for Marine Biomass Production and Co2 Remediation—a Review. J Appl Phycol, 1994. 6(1): p. 45-60.
2. Kraan, S., Algal Polysaccharides, Novel Applications and Outlook, in Carbohydrates—Comprehensive Studies on Glycobiology and Glycotechnology, C.-F. Chang, Editor. 2012, InTech.
3. Lee, K. Y. and D. J. Mooney, Alginate: properties and biomedical applications. Prog Polym Sci, 2012. 37(1): p. 106-26.
4. Evans, L. R. and A. Linker, Production and characterization of the slime polysaccharide of Pseudomonas aeruginosa. J Bacteriol, 1973. 116(2): p. 915-24.
5. Larsen, B. and A. Haug, Biosynthesis of alginate. 1. Composition and structure of alginate produced by Azotobacter vinelandii (Lipman). Carbohydr Res, 1971. 17(2): p. 287-96.
6. Rodde, R. S. H., K. Ostgaard, and B. Larsen, Chemical composition of protoplasts of Laminaria digitata (Phaeophyceae). Bot March, 1997. 40(5): p. 385-90.
7. Wong, T. Y., L. A. Preston, and N. L. Schiller, Alginate lyase: review of major sources and enzyme characteristics, structure-function analysis, biological roles, and applications. Annu Rev Microbiol, 2000. 54: p. 289-340.
8. Draget, K. I., O. Smidsrod, and G. Skja-Brwk, Alginates from algae, in Polysaccharides and polyamides in the food industry: properties, production, and patents, A. Steinbuchel and S. K. Rhee, Editors. 2005, Wiley-VCH: Weinheim.
9. Hamza, A., et al., Insight into the binding of the wild type and mutated alginate lyase (AlyVI) with its substrate: A computational and experimental study. Biochim Biophys Acta, 2011. 1814(12): p. 1739-47.
10. Kim, D. E., E. Y. Lee, and H. S. Kim, Cloning and characterization of alginate lyase from a marine bacterium Streptomyces sp. ALG-5. Mar Biotechnol (NY), 2009. 11(1): p. 10-6.
11. Park, H. H., et al., Cloning and Characterization of a Novel Oligoalginate Lyase from a Newly Isolated Bacterium Sphingomonas sp. MJ-3. Mar Biotechnol, 2011.
12. Tondervik, A., et al., Isolation of mutant alginate lyases with cleavage specificity for di-guluronic acid linkages. J Biol Chem, 2010. 285(46): p. 35284-92.
13. Uchimura, K., et al., Cloning and sequencing of alginate lyase genes from deep-sea strains of Vibrio and Agarivorans and characterization of a new Vibrio enzyme. Mar Biotechnol, 2010. 12(5): p. 526-33.
14. Liu, C.-L. Thermostable alginate lyase from Bacillus steraothermphilus. 1992. U.S. Pat. No. 5,139,945.
15. Alipour, M., Z. E. Suntres, and A. Omri, Importance of DNase and alginate lyase for enhancing free and liposome encapsulated aminoglycoside activity against Pseudomonas aeruginosa. J Antimicrob Chemother, 2009. 64(2): p. 317-25.
16. Mrsny, R. J., et al., Addition of a bacterial alginate lyase to purulent CF sputum in vitro can result in the disruption of alginate and modification of sputum viscoelasticity. Pulm Pharmacol, 1994. 7(6): p. 357-66.
17. Jones, C. S. and S. P. Mayfield, Algae biofuels: versatility for the future of bioenergy. Curr Opin Biotechnol, 2011.
18. Wargacki, A. J., et al., An engineered microbial platform for direct biofuel production from brown macroalgae. Science, 2012. 335(6066): p. 308-13.
19. Bjornsdottir, S. H., et al., Rhodothermus marinus: physiology and molecular biology. Extremophiles, 2006. 10(1): p. 1-16.
20. Jenkins, J., O. Mayans, and R. Pickersgill, Structure and evolution of parallel beta-helix proteins. J Struct Biol, 1998. 122(1-2): p. 236-46.
21. Karlsson, E. N., et al., The modular xylanase Xyn10A from Rhodothermus marinus is cell-attached, and its C-terminal domain has several putative homologues among cell-attached proteins within the phylum Bacteroidetes. FEMS Microbiol Lett, 2004. 241(2): p. 233-42.
22. Su, H., et al., Isolation and expression in Escherichia coli of hepB and hepC, genes coding for the glycosaminoglycan-degrading enzymes heparinase II and heparinase III, respectively, from Flavobacterium heparinum. Appl Environ Microbiol, 1996. 62(8): p. 2723-34.
23. Motejadded, H. and J. Altenbuchner, Construction of a dual-tag system for gene expression, protein affinity purification and fusion protein processing. Biotechnol Lett, 2009. 31(4): p. 543-9.
24. Bradford, M. M., A rapid and sensitive method for the quantitation of microgram quantities of protein utilizing the principle of protein-dye binding. Anal Biochem, 1976. 72: p. 248-54.
25. Laemmli, U. K., Cleavage of structural proteins during the assembly of the head of bacteriophage T4. Nature, 1970. 227(5259): p. 680-5.
26. Hreggvidsson, G. O., et al., Exploring novel non-Leloir beta-glucosyltransferases from proteobacteria for modifying linear (beta1->3)-linked gluco-oligosaccharide chains Glycobiology, 2011. 21(3): p. 304-28.
27. Jonsson, J. O., Beta-glucan transferases of family GH17 from Proteobacteria. 2010. M. Sc. Thesis. University of Iceland.

1. A recombinant construct comprising a DNA sequence comprising a coding region for a thermostable alginate lyase enzyme.

2. The recombinant construct of claim 1 wherein the thermostable alginate lyase is from Rhodothermus marinus.

3. The recombinant construct of claim 1 wherein the thermostable alginate lyase is selected from AlyRm1 (SEQ ID NO:1, SEQ IDNO:2), AlyRm2 (SEQ ID NO:3, SEQ ID NO:4), AlyRm3 (SEQ ID NO:5), and AlyRm4 (SEQ ID NO:6) from Rhodothermus marinus.

4. The recombinant construct of claim 1 comprising a sequence selected from SEQ ID NO:7, SEQ ID NO:8, SEQ ID NO:9, SEQ ID NO:10, SEQ ID NO:11, and SEQ ID NO:12.

5. The recombinant construct of claim 1 wherein the thermostable alginate lyase is from an organism selected from the genera of Alicyclobacterium, Ammonifex, Anerocellum, Anaerolinea, Anaerophaga, Anoxybacillus, Caldicellulosiruptor, Clostridium, Caldilinea, Caldisericum, Calditerrivibrio, Caloramator, Chloroacidobacterium, Carboxydothermus, Chloroflexus, Clostridium, Desulfotomaculum, Dictyoglomus, Exiguobacterium, Fervidobacterium, Geobacillus, Marinithermus, Marinitoga, Meiothermus, Oceanithermus, Paenibacillus, Petrotoga, Rhodothermus, Roseiflexus, Spirochaeta, Syntrophothermus, Thermacetogenium, Thermaerobacter, Thermanaerovibrio, Therminicola, Thermoanaerobacter, Thermoanaerobacterium, Thermobacillus, Thermobaculum, Thermobifida, Thermobispora, Thermodesulfatator, Thermodesulfobacterium, Thermodesulfobium, Thermodesulfovibrio, Thermomicrobium, Thermomonospora, Thermosediminibacter, Thermosipho, Thermosynecchococcus, Thermotoga, Thermovibrio, Thermovirga, Thermus and other genera of thermophilic organisms.

6. An isolated protein comprising an enzymatically active alginate lyase sequence having a thermostable alginate lyase activity.

7. The isolated protein of claim 6, having optimal activity at a temperature which is about 60° C. or higher.

8. The isolated protein of claim 6, where the protein is a fusion protein comprising additional His-tag.

9.-11. (canceled)

12. The isolated protein of claim 6 having a thermostable alginate lyase activity on alginate oligo/polysaccharides such that it preferentially cleaves M-G bonds and less preferentially G-G or M-M or G-M bonds.

13. The isolated protein of claim 6 having a thermostable alginate lyase activity on alginate oligo/polysaccharides such that it preferentially cleaves M-G or G-G bonds and less preferentially M-M or G-M bonds.

14. The isolated protein of claim 6 having a thermostable alginate lyase activity on alginate oligo/polysaccharides such that it preferentially cleaves M-G or G-G or M-M or G-M bonds in a random fashion.

15. The isolated protein of claim 6 having a thermostable alginate lyase activity on alginate oligo/polysaccharides such that it preferentially cleaves M-M in an Exo-fashion and yielding preferentially monosugars.

16. A method for carrying out an alginate degradation reaction comprising adding an alginate substrate to a reaction mixture, adding to the reaction mixture a protein an enzymatically active thermostable alginate lyase protein, and incubating the reaction mixture at a temperature of about 50° C. or higher.

17. The method of claim 16, wherein the reaction mixture is incubated at a temperature of about 60° C. or higher.

18. (canceled)

19. The method of claim 16 wherein the thermostable alginate lyase enzyme used is isolated from a microbial production host comprising a recombinant construct comprising a DNA sequence comprising a coding region for a thermostable alginate lyase enzyme.

20. The method of claim 19 wherein the coding region for a thermostable alginate lyase enzyme is from Rhodothermus marinus.

21. The method of claim 19 wherein the coding region for a thermostable alginate lyase enzyme comprises a sequence selected from SEQ ID NO:7, SEQ ID NO:8, SEQ ID NO:9, SEQ ID NO:10, SEQ ID NO:11, and SEQ ID NO:12.

22. The method of claim 19 wherein the coding region for a thermostable alginate lyase enzyme is selected from the group consisting of the genera of Alicyclobacterium, Ammonifex, Anerocellum, Anaerolinea, Anaerophaga, Anoxybacillus, Caldicellulosiruptor, Clostridium, Caldilinea, Caldisericum, Calditerrivibrio, Caloramator, Chloroacidobacterium, Carboxydothermus, Chloroflexus, Clostridium, Desulfotomaculum, Dictyoglomus, Exiguobacterium, Fervidobacterium, Geobacillus, Marinithermus, Marinitoga, Meiothermus, Oceanithermus, Paenibacillus, Petrotoga, Rhodothermus, Roseiflexus, Spirochaeta, Syntrophothermus, Thermacetogenium, Thermaerobacter, Thermanaerovibrio, Therminicola, Thermoanaerobacter, Thermoanaerobacterium, Thermobacillus, Thermobaculum, Thermobifida, Thermobispora, Thermodesulfatator, Thermodesulfobacterium, Thermodesulfobium, Thermodesulfovibrio, Thermomicrobium, Thermomonospora, Thermosediminibacter, Thermosipho, Thermosynecchococcus, Thermotoga, Thermovibrio, Thermovirga, Thermus and other genera of thermophilic organisms.

23.-25. (canceled)

26. The method of claim 16, wherein the thermostable alginate lyase protein comprises a sequence selected from the group consisting of SEQ ID NO:1, SEQ ID NO:2, SEQ ID NO:3, SEQ ID NO:4, SEQ ID NO:5, and SEQ ID NO:6.

27. The method of claim 16, wherein the alginate lyase enzyme is comprised in a fusion protein comprising a His-tag and said enzymatically active thermostable alginate lyase protein domain.

28. The method according to claim 16 wherein the thermostable alginate lyase enzyme has activity on alginate oligo/polysaccharides such that it preferentially cleaves M-G bonds and less preferentially G-G or M-M or G-M bonds.

29. The method according to claim 16 wherein the thermostable alginate lyase enzyme has activity on alginate oligo/polysaccharides such that it preferentially cleaves M-G or G-G bonds and less preferentially M-M or G-M bonds.

30. The method according to claim 16 wherein the thermostable alginate lyase enzyme has activity on alginate oligo/polysaccharides such that it preferentially cleaves M-G or G-G or M-M or G-M bonds in a random fashion.

31. The method according to claim 16 wherein the thermostable alginate lyase enzyme has activity on alginate oligo/polysaccharides such that it preferentially cleaves M-M in an Exo-fashion and yielding preferentially monosugars.

32. The method according to claim 16 wherein the thermostable alginate lyase enzyme used has optimum activity higher than at 60° C.

33. The method of claim 16 wherein the substrate is an alginate oligosaccharide.

34. The method of claim 16 wherein the substrate is an alginate polysaccharide.

35. The method of claim 16 wherein the substrate comprises a polysaccharide derived from macroalgae.

36. The method of claim 16 wherein the reaction mixture contains an enzymatically active thermostable alginate lyase of more than one protein type.

37.-39. (canceled)

40. The method of claim 16 wherein the enzymatically active thermostable alginate lyase is immobilized, during the process of alginate degradation.

41. The method of claim 16 wherein the substrate comprises a polysaccharide derived from macroalgae from the genera of Microcystis, Ascophyllum, Laminaria, Ecklonia or Sargassum.

42. The method of claim 16 wherein the substrate comprises a polysaccharide derived from bacteria.

43. The method of claim 16 wherein the substrate comprises a polysaccharide derived from bacteria from the genera of Pseudomonas and Azotobacter.

44. (canceled)

45. The method of claim 16 wherein the produced degradation products are at least 50% (unsaturated) mono-uronates.

46.-49. (canceled)

50. The method of claim 44 further comprising a step of fermenting the degradation products.

51. The method of claim 44 further comprising a step of fermenting the produced degradation products to alcohols.

52.-56. (canceled)

57. An isolated polynucleotide comprising a sequence coding for a thermostable alginate lyase selected from the group consisting of AlyRm1 depicted in SEQ ID NO:1, AlyRm2 depicted in SEQ ID NO:2, AlyRm3 depicted in SEQ ID NO:3, and AlyRm4 depicted in SEQ ID NO: 4.

58. The isolated polynucleotide of claim 57, comprising a sequence selected from SEQ ID NO:5, SEQ ID NO:6, SEQ ID NO:7, and SEQ ID NO:8.


SEQUENCE LISTING (23 sequences)

SEQ ID NO: 1 - protein, 564 aa, Rhodothermus marinus
MQAQAVRYVTTPEELQVAIQAAQPGDTIVMADGTWRDVAIVFEANGAPGDTITLRAETPGRVVLTGSSRLRIGGAYLKVEGLRFENGALPDGEGVIEFRANGRHAFHSRLTNTAIVNYNPPGRETEYKWVSLYGRFNRVDHCYFAGKTHLGALLVVWLQDPPNDAPPQHRFDHNYFGPRPELGENGAEIIRIGTSARSMQEARVVVERNLFLETNGEIEIISNKSGGNIYRGNTFRRCRGTLTLRHGNGALVEGNFFFGEGIAGTGGVRIIGEDHRVINNYFQDLTGTGYYAAVSVVQGVPDSPLNRYFQVKRAVIAHNTFVNTERSFEIGIGASPDQSLPPEDLTIVNNVVQTRPGAPIVTTHLEPIGNTVWAGNIFYGKPGSFPEGAATFAHPELVRGDDGLYRPAPTSPLIDAAHPDFAPAVDMDGQPRSDRAPDVGADEVSDAPVRWKPLTPADVGPDWLREANIEVKFGGRPSRLAPHTSGFPFEGVTDMSFYVERPSHVRISMFDLSGRRVMTVFDERVEEGAHVFRLDGSHLPTGTYLLVMETDCNERDYRLVTIRR

SEQ ID NO: 2 - protein, 474 aa, Rhodothermus marinus
MQAQAVRYVTTPEELQVAIQAAQPGDTIVMADGTWRDVAIVFEANGAPGDTITLRAETPGRVVLTGSSRLRIGGAYLKVEGLRFENGALPDGEGVIEFRANGRHAFHSRLTNTAIVNYNPPGRETEYKWVSLYGRFNRVDHCYFAGKTHLGALLVVWLQDPPNDAPPQHRFDHNYFGPRPELGENGAEIIRIGTSARSMQEARVVVERNLFLETNGEIEIISNKSGGNIYRGNTFRRCRGTLTLRHGNGALVEGNFFFGEGIAGTGGVRIIGEDHRVINNYFQDLTGTGYYAAVSVVQGVPDSPLNRYFQVKRAVIAHNTFVNTERSFEIGIGASPDQSLPPEDLTIVNNVVQTRPGAPIVTTHLEPIGNTVWAGNIFYGKPGSFPEGAATFAHPELVRGDDGLYRPAPTSPLIDAAHPDFAPAVDMDGQPRSDRAPDVGADEVSDAPVRWKPLTPADVGPDWLREANIEVKFG

SEQ ID NO: 3 - protein, 581 aa, Rhodothermus marinus
MTIGAVVVEDTTPPMVYTVRTAEELKSVLRGELRPGDIVEVEDGVYDTGGGITIEASGTETKPIIIRAKNIGLAELTGKTYFTFRKSSYIILEGFKFTSNVYTAVKLEACHHIRITRNIFQLDETGRESSKWIVVGGYYADPSLLSHHNRIDHNIFRDKQTLGNFITIDGGDVVSQHDRIDHNYFYNIGPRAENEKEAIRVGWSELSLTDGYTVIEYNLFERCDGDPEIVSIKSSKDTVRYNTFRASQGSLTLRHGNGSVVYGNFFLGEGREGTGGVRVYAKDHKIYNNYFEGLTGSVWDAAITLTNGDTDEGSLSAHWRVQNVLIAHNTLVNNYSNIEIGYARSDNSWKKEPRNVQIINNLVVAGELTNRDLITIYTEPTDFVWAGNIMYPKTGYGLGITADPAEIFVADPLLEENNGLWVLSAQSPAVDAGSSLNFSIMEDFQGQPRDALPDVGADELSSAPVLRRPLQPEDVGPFASDSISTSVEVRSHRPAQTVLKGFPNPFITSTVLAFSLPERSQITLFVYDMLGREVARLYDGELEAGSYRLVWQPEDELASGIYLVVLTTDRGTAAYKLALIR

SEQ ID NO: 4 - protein, 489 aa, Rhodothermus marinus
MTIGAVVVEDTTPPMVYTVRTAEELKSVLRGELRPGDIVEVEDGVYDTGGGITIEASGTETKPIIIRAKNIGLAELTGKTYFTFRKSSYIILEGFKFTSNVYTAVKLEACHHIRITRNIFQLDETGRESSKWIVVGGYYADPSLLSHHNRIDHNIFRDKQTLGNFITIDGGDVVSQHDRIDHNYFYNIGPRAENEKEAIRVGWSELSLTDGYTVIEYNLFERCDGDPEIVSIKSSKDTVRYNTFRASQGSLTLRHGNGSVVYGNFFLGEGREGTGGVRVYAKDHKIYNNYFEGLTGSVWDAAITLTNGDTDEGSLSAHWRVQNVLIAHNTLVNNYSNIEIGYARSDNSWKKEPRNVQIINNLVVAGELTNRDLITIYTEPTDFVWAGNIMYPKTGYGLGITADPAEIFVADPLLEENNGLWVLSAQSPAVDAGSSLNFSIMEDFQGQPRDALPDVGADELSSAPVLRRPLQPEDVGPFASDSISTSVEV

SEQ ID NO: 5 - protein, 855 aa, Rhodothermus marinus
MQNPYETYTGFTVPTEAVLPDSEVHPSLWFSAEELATIRARWQDPAYAELVDEIKRDIRDFKNRNPESTEPGERARMAKTLAFAWLMENDVVALVKALATLEVAYDNVPQTYDSGVFDGEYDEIYRATWLQNYCAAYDWLYDQLGSQLEAELRAKLVAEAQLLYTYMNQYAPRPHNHRSKPAYALGTAALTLSSHPNAAQWLSFALDRQNSVTKYMFSHEGVYREGPHYYVFTLVNAIPFLWHYLHVSGANLFPYYQPAFEWPIRIRNSRGWMPNIEDGFMKPAPTHAVAAAYRDTPTLLHSSAPLAEILQWNWQTTRFFTQNYTGATNDVTWEIDVLLSWDASIPATPPDVSPTQVLQSGQVAFRNAWSDVGESSRYLLFHGVASADNHDHPDHLSYVVDAANTTLAVDAGYGPEGSSDDRRSWYTSPQAHNTVTVNGFPLVDYSTARNEGPRLRHALDTPFYDFAEMQARSQGVAGGAEVRRGIAFPEERFWVVYDLGSSDNEASYQVHLHGRGTFARNGSWLTWTAQPDTYGEGARLHAAFAGNRTLTISENTGWTSLYWGHEETQTYVSVRQTATDPVFLHVLYPTPLNGTPPALVDRSGSGIVSLELTEDAGITNVAVQRDQVLRTAGPLATDAIFAWTRRVQGNIVQFALTEGRELRWGGRLLLSASDTLTVAVNRSNPSRQLLYVEPFTGQAELTLRLLPDTATPLSVTLDGQPLAFETPEQGTVRFQLSGDRLGAGSVIVVTTNVASAAEPHEAATAGFMIEGPYPNPTSGPMHLRLVLARPAHVRAVLYDVLGRRLATLWDGAVGAGQTELTWDVGRLLRQKLTPGPYLIEVEADGARRVVRGLAF

SEQ ID NO: 6 - protein, 722 aa, Rhodothermus marinus
MLEVLAQPRHPRVFISAEEAAALRENAERYTLLRTTLARVRAEMEEVLAKPIEIPPPGEAGGYEHERHKQNYREMFKAGLLYQITGDERYARFVRDMLMGYAAMYPELGPHPRSERQIPGKLFHQLLNECVWLVHTIVAYDAIYNWLSEADRAHIEASVFRPMASWIMNEGAYEFNRIHNHGTWAVAAVGMTGYVIGEPDWVEKALYGSNKEGKSGFYAQLDQLFSPDGYYMEGPYYARYALWPFFFFAEAIERYEPERGIYAYRDSILKKALYATVQTAFPDGVLPPLNDASLTMDIRAPGVVLATDVVFARYEGDPALLGVAHEQGQVVLNGAGLAVAKAYDEASEVPRMTWPSVEFTDGADGRRGGIGFLRTGVGRDQTVVLMKYGVHGLGHGHFDKLHFMLYDNGRQVIRDYGFARFINVEPKYGGRYLPENESYAKQTIAHNTVVVDETSQNRADRDEAGAMSGQRHFFDGRGGPVQAMSARANGYWPGVNMQRTLLLVEDARLDYPVVVDLFRVTADTMHQYDYPLHFLGQPIWDNLGIRGYPDQLRPLGTDFGYQHIWEEARARTDSVVQFTWLDGSRYYTWTVAAAPATEVILGRIGAGDPRFNLRREPMILVRRRGRDHLFASVIEPHGYFNEAREISLRARPQVQAVRVIGHSEAASVVEVVGKGGWTWTIMVTNGPADPDARHTVTFGNRTFSWTGNYAVEGVVVASDSRR

SEQ ID NO: 7 - DNA, 1693 bp, Rhodothermus marinus
atggcaggcccaggccgtccgttacgtgaccacgcccgaagagctgcaggtcgccattcaggccgcacagcctggcgacaccatcgttatggccgacggcacctggcgagatgttgcgatcgtctttgaggccaacggggccccgggcgacaccatcacgctgcgggccgagacgcccggtcgcgtggtgctgacgggcagttcccgacttcgcatcgggggggcctacctcaaggtggaaggactccgcttcgaaaacggcgcgctgcccgacggcgaaggtgtgatcgaattccgggctaacggccggcatgcctttcacagccgcctgaccaacacggccatcgtcaactacaacccgcccggcagggagaccgagtacaaatgggtttccctgtacgggcgcttcaaccgggtcgatcactgctatttcgccggaaagacgcatctgggggctctgctggtcgtgtggctgcaggatccgcccaacgatgcgccgccccagcaccgattcgaccataactacttcgggccacgtccggagctgggggaaaacggcgccgagatcatccggatcggcaccagtgctcgctcgatgcaggaggcccgcgtcgtggtggagcgcaacctctttctggagacgaacggcgaaatcgaaatcatctccaacaaatcgggcggcaacatctatcggggcaataccttccggcggtgccgggggacgctcacgctgcgtcacggcaacggcgcgctcgtcgaaggcaacttcttcttcggggaaggaatcgcgggtacgggcggcgtgcgcatcatcggcgaagaccaccgcgtgatcaacaactactttcaggacctgaccgggaccggctactatgcggcggtctcggtggtgcagggcgtgccagactcgccgctcaaccgctacttccaggtcaaacgcgcggtgatcgcccacaacaccttcgtcaataccgagcgtagcttcgaaatcggcatcggggccagcccggaccagtcgctgccgcccgaagacctgaccatcgtaaacaacgtggtgcagacgcgaccgggcgctccgatcgtaacaacgcacctggagccgatcggaaataccgtctgggccggtaacatcttctatggcaaacccggatcattcccggaaggagcggcgacgttcgcccatccggaactggtccggggcgacgatggcctctaccgcccggcgcctaccagcccgctcatcgatgcggcgcacccggacttcgcccccgcagtggatatggacgggcagccgcgtagcgatcgggcgcccgacgtgggggccgacgaggtttcggacgcccccgtacgctggaaaccgctcacacccgccgacgtggggcccgactggctgcgggaggcaaacatcgaagtgaagttcggaggaaggccgtccagactggcaccgcatacctccggctttcccttcgaaggcgtgaccgacatgagcttctacgtggagcggccgtcccatgtgcggatcagcatgttcgatctgagcgggcgcagggtgatgaccgtcttcgatgagcgcgtcgaggaaggagcccacgtgtttcgcctcgacgggagccacttgcccaccgggacctacctgctggtgatggagaccgactgcaacgagcgcgactatcggctggttaccatacgacgc

SEQ ID NO: 8 - DNA, 1422 bp, Rhodothermus marinus
atgcaggcccaggccgtccgttacgtgaccacgcccgaagagctgcaggtcgccattcaggccgcacagcctggcgacaccatcgttatggccgacggcacctggcgagatgttgcgatcgtctttgaggccaacggggccccgggcgacaccatcacgctgcgggccgagacgcccggtcgcgtggtgctgacgggcagttcccgacttcgcatcgggggggcctacctcaaggtggaaggactccgcttcgaaaacggcgcgctgcccgacggcgaaggtgtgatcgaattccgggctaacggccggcatgcctttcacagccgcctgaccaacacggccatcgtcaactacaacccgcccggcagggagaccgagtacaaatgggtttccctgtacgggcgcttcaaccgggtcgatcactgctatttcgccggaaagacgcatctgggggctctgctggtcgtgtggctgcaggatccgcccaacgatgcgccgccccagcaccgattcgaccataactacttcgggccacgtccggagctgggggaaaacggcgccgagatcatccggatcggcaccagtgctcgctcgatgcaggaggcccgcgtcgtggtggagcgcaacctctttctggagacgaacggcgaaatcgaaatcatctccaacaaatcgggcggcaacatctatcggggcaataccttccggcggtgccgggggacgctcacgctgcgtcacggcaacggcgcgctcgtcgaaggcaacttcttcttcggggaaggaatcgcgggtacgggcggcgtgcgcatcatcggcgaagaccaccgcgtgatcaacaactactttcaggacctgaccgggaccggctactatgcggcggtctcggtggtgcagggcgtgccagactcgccgctcaaccgctacttccaggtcaaacgcgcggtgatcgcccacaacaccttcgtcaataccgagcgtagcttcgaaatcggcatcggggccagcccggaccagtcgctgccgcccgaagacctgaccatcgtaaacaacgtggtgcagacgcgaccgggcgctccgatcgtaacaacgcacctggagccgatcggaaataccgtctgggccggtaacatcttctatggcaaacccggatcattcccggaaggagcggcgacgttcgcccatccggaactggtccggggcgacgatggcctctaccgcccggcgcctaccagcccgctcatcgatgcggcgcacccggacttcgcccccgcagtggatatggacgggcagccgcgtagcgatcgggcgcccgacgtgggggccgacgaggtttcggacgcccccgtacgctggaaaccgctcacacccgccgacgtggggcccgactggctgcgggaggcaaacatcgaagtgaagttcgga

SEQ ID NO: 9 - DNA, 1743 bp, Rhodothermus marinus
atgacgatcggtgcggtggtggtggaagataccacgccgccgatggtctatacggtacgcaccgccgaggaattgaagtcggtgctgcggggtgaactccggcccggggatattgtggaagtggaggacggcgtttacgacacaggtggggggatcaccatcgaagccagtggcaccgaaacgaaaccgatcatcatccgggcaaagaacatcgggctggcggagttgaccggtaagacgtactttaccttccgaaaaagctcctacatcattctggaaggcttcaagttcacctccaacgtttacacagcggtcaaactggaagcctgtcatcacatccgtatcacgcgcaacatctttcagctggatgagacgggacgcgagagtagtaagtggattgtggtcggagggtattatgccgatcccagcctgttaagccatcacaaccgaatcgatcacaacatcttccgagataaacagaccctgggtaacttcatcacgattgacgggggtgatgttgtatcgcagcacgatcgcatcgatcacaactatttctacaacattggtcctcgtgcggaaaatgagaaagaggccattcgggtgggatggagtgaactctctctgaccgatggctacaccgtaatcgagtacaatctgtttgagcgctgtgatggcgatccggaaattgtgtcgatcaaaagttcgaaagacacggtccgatacaatacgttccgggcgagccagggctcactgaccctgcgccatggcaacggatcggtggtgtatggtaatttcttcctgggcgaaggcagagaaggtaccggcggcgttcgcgtttacgcgaaagaccacaagatttacaacaactacttcgaggggttgaccggtagcgtctgggatgcagccattacgctcaccaacggtgacaccgacgaggggtccctgagcgcgcactggcgcgtccagaatgtgctgatcgcgcacaacacgctggtcaacaattactccaacatcgaaatcggctacgcgcgttccgacaactcctggaagaaggagccgaggaacgtgcagatcatcaacaacctggtggtggcgggagagctgaccaacagagatctgatcacgatttatacggagccgacagattttgtgtgggcgggcaacatcatgtatccgaagacggggtatggcctgggcataacggccgaccctgcggaaattttcgtggcagatccgttactggaagaaaacaatggtctctgggttctcagtgcgcagagtccggcggttgatgcgggcagcagtctcaacttttccatcatggaagatttccagggccagcccagagacgctctgcccgatgttggcgcagatgaactgtcttcagcgcctgttttacgtcggccgttgcagccagaagacgtagggccgtttgcttcggatagtatcagcacgagcgttgaagtacgaagccatcggcctgcacagacggtgctgaaaggctttcccaacccgtttatcacttcgaccgtactggctttcagtctgcctgagcggtcacagataaccctgtttgtttatgatatgctgggacgtgaggtggcgcgcctctatgacggggaactggaggccggaagctatcgactggtatggcagcctgaagacgaactggcatcgggtatatatctggttgtgttgaccacggatcgaggaacagccgcttacaaacttgccctgatcagg

SEQ ID NO: 10 - DNA, 1467 bp, Rhodothermus marinus
atgacgatcggtgcggtggtggtggaagataccacgccgccgatggtctatacggtacgcaccgccgaggaattgaagtcggtgctgcggggtgaactccggcccggggatattgtggaagtggaggacggcgtttacgacacaggtggggggatcaccatcgaagccagtggcaccgaaacgaaaccgatcatcatccgggcaaagaacatcgggctggcggagttgaccggtaagacgtactttaccttccgaaaaagctcctacatcattctggaaggcttcaagttcacctccaacgtttacacagcggtcaaactggaagcctgtcatcacatccgtatcacgcgcaacatctttcagctggatgagacgggacgcgagagtagtaagtggattgtggtcggagggtattatgccgatcccagcctgttaagccatcacaaccgaatcgatcacaacatcttccgagataaacagaccctgggtaacttcatcacgattgacgggggtgatgttgtatcgcagcacgatcgcatcgatcacaactatttctacaacattggtcctcgtgcggaaaatgagaaagaggccattcgggtgggatggagtgaactctctctgaccgatggctacaccgtaatcgagtacaatctgtttgagcgctgtgatggcgatccggaaattgtgtcgatcaaaagttcgaaagacacggtccgatacaatacgttccgggcgagccagggctcactgaccctgcgccatggcaacggatcggtggtgtatggtaatttcttcctgggcgaaggcagagaaggtaccggcggcgttcgcgtttacgcgaaagaccacaagatttacaacaactacttcgaggggttgaccggtagcgtctgggatgcagccattacgctcaccaacggtgacaccgacgaggggtccctgagcgcgcactggcgcgtccagaatgtgctgatcgcgcacaacacgctggtcaacaattactccaacatcgaaatcggctacgcgcgttccgacaactcctggaagaaggagccgaggaacgtgcagatcatcaacaacctggtggtggcgggagagctgaccaacagagatctgatcacgatttatacggagccgacagattttgtgtgggcgggcaacatcatgtatccgaagacggggtatggcctgggcataacggccgaccctgcggaaattttcgtggcagatccgttactggaagaaaacaatggtctctgggttctcagtgcgcagagtccggcggttgatgcgggcagcagtctcaacttttccatcatggaagatttccagggccagcccagagacgctctgcccgatgttggcgcagatgaactgtcttcagcgcctgttttacgtcggccgttgcagccagaagacgtagggccgtttgcttcggatagtatcagcacgagcgttgaagta

SEQ ID NO: 11 - DNA, 2565 bp, Rhodothermus marinus
atgcagaacccttatgagacttacacgggctttacggtcccgaccgaagccgtccttccggacagcgaagtgcatccctcgctgtggttttcggccgaagagcttgccacgatccgggctcgctggcaggacccggcttacgccgagctggtggacgaaatcaaacgggacatccgggacttcaaaaatcgcaaccctgaatccaccgagcccggcgaacgggcacggatggccaagacgctggccttcgcctggctgatggaaaacgatgtcgtggcgctggtaaaagcgcttgcaacgctggaggtggcctacgacaacgtaccccagacgtacgattcaggcgtgttcgatggcgaatacgacgagatctaccgggccacgtggttgcagaactactgcgccgcctacgactggctgtacgaccagctcgggtcccagctggaggccgaattacgcgccaaactggtggccgaggcgcaactgctctacacctacatgaaccagtacgcgccaaggccgcacaaccaccggtccaagccggcctatgcgctcgggacggcggcgctgacactgtcgagccatccgaatgcggcgcagtggctctcgtttgcgctcgaccggcagaacagcgtgacgaagtacatgttcagccacgagggcgtctatcgagaagggcctcactattatgtcttcacgctggtcaacgcgattccttttctctggcattacctgcacgtgtcgggtgcgaaccttttcccgtactatcagccggccttcgaatggcccatccgtatccgaaacagccgagggtggatgcccaacatcgaagacggcttcatgaagcccgcccccacgcatgccgtggccgcagcctatcgcgacacgccgaccctgctgcacagcagcgccccgctggccgaaattctacagtggaactggcagaccacccgctttttcacgcagaactacaccggcgccacgaacgacgtcacctgggagatcgacgtgctgctctcctgggacgcgagtattccggcaacgcctccggacgtctcgccgacgcaggtgttgcagagcgggcaggtggcctttcgcaatgcctggagcgatgtaggggagtcgtcgcgctatctactgttccatggcgtggcctcggccgacaaccacgaccaccctgatcatctctcgtacgtggtggatgcggccaacacgacgctggccgtggatgccggttacgggccggagggctccagcgacgaccggcgtagctggtacacgtcgcctcaggcccacaacacggtcacggtcaacggtttcccgctggtcgattacagcacggcgcggaacgaaggtccacgtctgcggcacgcgctcgacacgccgttttacgacttcgccgaaatgcaggcccgcagccagggcgtggccggcggcgccgaagtacggcgcggcattgcgtttcctgaagaacgtttctgggtggtgtacgacctgggctcctcggacaacgaggccagctaccaggtgcacctgcacgggcgggggacgtttgcgcgcaacggttcatggctgacctggaccgcccagcctgacacctacggcgagggcgcccgcctgcatgcggcgttcgccggcaaccgcacgctgacgatttcggagaacaccggctggacgagtctctactgggggcatgaggagacgcagacctacgtttccgtgcggcagacggccaccgatccggtctttctgcacgtgctctatccgactccgttgaacggaacgcctccggcgctggtggaccggagcggctccggcatcgtcagtctggagctgaccgaagacgcgggcatcaccaacgtggccgtgcagcgagatcaggtgctgcgaacggccggtccgctggcgaccgacgcgatttttgcctggacccggcgcgtgcaggggaacatcgtgcagttcgctctgaccgaagggcgagaactccggtggggagggcgcctgctgctttcggccagcgacacgctgacggtggccgtcaatcgctccaatccgtcgcgtcagttgctgtatgtggagccgttcaccggacaggccgagttgacgcttcggctgctgcccgacacggccacgccgctttccgtcacgctcgacggacagccgctggccttcgagacgccggagcagggcacggtgcgcttccagctcagcggcgatcggctcggagccggcagcgtcattgtggtaacaacgaacgtggcctcggccgccgagccccatgaggcggccaccgcaggtttcatgatagagggaccctatcccaaccccacgtcggggccgatgcaccttcggcttgtactggcccgaccggcgcatgtacgggcggtgctgtacgacgtgctgggacgtcgcctggcgacgctgtgggatggggcggtgggagccggacagaccgaattgacctgggatgtgggacgcctgttgcgacagaaactgacgccagggccctatctgatcgaggtggaggccgacggcgcgcgcagggtagtccggggtctggcattt

SEQ ID NO: 12 - DNA, 2166 bp, Rhodothermus marinus
atgctggaagtgctcgcgcagccgcggcatccccgtgtgttcatctcggccgaagaggcggcggcacttcgggagaatgccgagcgctacacgctgctcaggaccacgctggcgcgcgtgcgggccgagatggaagaggtcctggcgaagcccatcgaaattccgccgcccggcgaagccggtggttacgagcacgagcggcacaagcagaactaccgcgaaatgttcaaagccgggctgctctaccagatcaccggcgacgaacgctatgcccggttcgtgcgggatatgctgatgggctatgcggccatgtatcccgagctggggccgcatccgcgcagcgagcgccagattcccggcaagctcttccaccagctgctcaacgagtgcgtctggctggtgcacaccattgtggcctacgacgcgatctacaactggctgagcgaggccgatcgggcccacatcgaggccagcgtgtttcgtccgatggcctcctggatcatgaacgaaggggcctacgagttcaaccgcatccacaaccacggcacctgggcggtagccgcggtgggcatgaccggctacgtgatcggcgagccggactgggtggaaaaggcgctttacggcagcaataaagagggaaaaagcgggttttatgcgcagctcgatcagctcttctcaccggacggctactacatggaaggcccctattacgcccgctatgcgctctggcctttcttcttctttgccgaggcgatcgagcggtacgagcccgagcgcggcatctacgcctatcgcgacagcatcctgaaaaaagccctttatgccacggtgcagaccgcctttccggatggcgtcctgccgccgcttaacgatgcctcgctcacgatggacattcgggcgccgggcgtcgtgctggccaccgatgtggtgtttgcccgctacgagggcgatccggccctgctgggcgtggcgcacgaacagggccaggtggtgctcaacggcgcggggctggccgtggcgaaagcctacgacgaggcgtccgaagtgccccggatgacgtggcccagtgtcgagttcaccgacggggccgacggccggcgaggtggtatcggctttctgcgtaccggcgtgggccgcgaccagaccgtcgtgctcatgaagtacggcgtgcacggactggggcacggccacttcgacaagctgcacttcatgctctacgacaacggccgccaggtcatccgagactacggctttgcccggttcatcaacgtggaacccaagtacggcggccgctacctcccggaaaacgaaagctacgccaagcagaccatcgctcataacacggtggtggtggacgagaccagccagaaccgggccgatcgggacgaggccggggccatgtcgggccagcggcactttttcgacggcaggggcggccccgtacaggccatgagtgcgcgtgccaacggctactggccgggcgtgaacatgcagcggacgctgctgctcgtcgaagatgcgcggctggactatcccgtggtggtggacctcttccgggtgacggccgacaccatgcaccagtacgactatcccctgcactttctggggcaacccatctgggacaacctgggcattcggggctaccccgatcagctcaggccgctggggaccgacttcggctaccagcacatctgggaggaggcgcgagcgcgcaccgatagcgtcgtgcagtttacctggctcgatggctcgcgctactacacctggacggtggcggccgccccggccacagaggtcatcctgggacgcatcggcgctggcgatccccgcttcaatctacgccgggaaccgatgattctggtgcgccgccgcggccgggaccatctgttcgccagcgtcatcgagccgcatggttacttcaacgaggcgcgcgagatcagcctaagggccaggcctcaggtgcaggcggtacgggtgatcggccacagcgaggcggccagtgtggtggaggtggtcgggaagggcggctggacctggaccatcatggtgacgaacgggccggccgatcctgacgcccgccataccgtcacgttcggcaaccgcaccttctcgtggacgggcaattatgcggtggagggcgtggtcgttgccagtgattcccgccgt

SEQ ID NO: 13 - DNA, 27 bp, Rhodothermus marinus
aaaagatctcaggccgtccgttacgtg

SEQ ID NO: 14 - DNA, 30 bp, Rhodothermus marinus
aaaaagcttcagcgtcgtatggtaaccagt

SEQ ID NO: 15 - DNA, 35 bp, Rhodothermus marinus
aaaaagcttcatccgaacttcacttccgatgtttg

SEQ ID NO: 16 - DNA, 29 bp, Rhodothermus marinus
aaaaaaggatccacgatcggtgcggtggt

SEQ ID NO: 17 - DNA, 30 bp, Rhodothermus marinus
aaaaaaaagcttacctgatcagggcaagtt

SEQ ID NO: 18 - DNA, 27 bp, Rhodothermus marinus
aaaggatccacgatcggtgcggtggtg

SEQ ID NO: 19 - DNA, 32 bp, Rhodothermus marinus
aaaaagcttatacttcaacgctcggtgctgat

SEQ ID NO: 20 - DNA, 33 bp, Rhodothermus marinus
aaaggatcccagaacccttatgagacttacacg

SEQ ID NO: 21 - DNA, 30 bp, Rhodothermus marinus
aaaaagcttctaaaatgccagaccccggac

SEQ ID NO: 22 - DNA, 29 bp, Rhodothermus marinus
aaaggatccctggaagtgctcgcgcagcc

SEQ ID NO: 23 - DNA, 31 bp, Rhodothermus marinus
aaaaagcttaacggcgggaatcactggcaac